(19) United States
(12) Patent Application Publication
(10) Pub. No.: US 2004/0242469 A1
(43) Pub. Date: Dec. 2, 2004

(54) Angiogenesis and cardiac tissue engineering with peptide hydrogels and related compositions and methods of use thereof

(76) Inventors: Richard T. Lee, Weston, MA (US); Roger D. Kamm, Weston, MA (US); Daria Narmoneva, Brighton, MA (US); Shuguang Zhang, Lexington, MA (US)

Correspondence Address: Choate, Hall & Stewart, Exchange Place, 53 State Street, Boston, MA, 02109, US

(21) Appl. No.: 10/431,000

(22) Filed: May 7, 2003

Related U.S. Application Data
(60) Provisional application No. 60/380,234, filed on May 13, 2002.

(51) Int. Cl. A61K 38/17

(52) U.S. Cl. 514/12

(57) ABSTRACT

The invention provides compositions comprising self-assembling peptide hydrogels and cardiovascular system cells, particularly endothelial cells, cultured in or on the gels. According to certain embodiments of the invention the endothelial cells form capillary-like structures. The endothelial cell-gel matrix serves as a pre-vascularized scaffold that may be used to culture additional cell types. Formation of mature blood vessels in or on the scaffold involves addition of smooth muscle cells and/or fibroblasts. Compositions of the invention may be used for cell culture and for administration to a subject to treat a variety of conditions including, but not limited to, myocardial dysfunction or damage.

### CROSS-REFERENCE TO RELATED APPLICATION

This application claims priority to U.S. Provisional Patent Application Ser. No. 60/380,234, filed May 13, 2002, which is incorporated herein by reference.

### BACKGROUND OF THE INVENTION

Various forms of heart disease such as congestive heart failure are leading causes of morbidity and mortality in the United States and are increasing throughout the world. The dominant cause of heart failure is loss and/or reduced function of myocardium, which may be due to any of a number of causes such as coronary artery disease. Unlike hepatocytes, which can regenerate after liver injury, the limited regeneration potential of cardiomyocytes means that once myocardial tissue is lost or damaged, there is normally little scope for regeneration of functional myocardium in vivo.

A number of medications are available that enhance the function of remaining viable cardiac tissue, but their efficacy is limited. While transplantation from human donors (either live or cadaveric) has enjoyed significant success, the severe shortage of donors, the complexity of harvesting organs and delivering them to the recipient, and the potential for transmission of infectious agents are significant shortcomings of this approach. Another approach for the treatment of cardiac dysfunction involves the transplantation of various cell types into the heart. For example, implantation of skeletal muscle cells, bone marrow cells, embryonic cardiac myocytes, and myoblasts have been reported to stimulate revascularization of ischemic heart tissue and enhance cardiac function in model systems (Li et al. 1996; Sakai et al. 1999; Scorsin et al. 1997; Taylor et al. 1998). However, the ability of isolated cells to recapitulate the complex three-dimensional architecture, cell-cell interactions, and functional activity of normal myocardium in vivo remains unclear.

Tissue engineering is a promising and actively developing area of research that seeks to develop methods for culturing replacement tissues and organs in the laboratory. The general strategy for producing replacement tissues utilizes mammalian cells that are introduced to an appropriate substrate for cell culture. Cells can be obtained from the intended recipient (e.g., from a biopsy), in which case they are often expanded in culture before being used to seed the substrate. Cells can also be obtained from other sources (e.g., established cell lines). After seeding, cell growth is generally continued in the laboratory and/or in the patient following implantation of the engineered tissue. Tissue engineering of cardiac tissue offers an attractive alternative to the other available means of treating cardiac dysfunction.

A variety of substrates have been explored for tissue engineering purposes. Many previous efforts to develop improved cell culture systems have involved the use of materials such as proteins and peptides obtained from animal or human sources. Such materials have a number of disadvantages as compared with synthetic materials. For example, they present an increased risk for the transmission of disease. In addition, it can be difficult to ensure that different preparations of material have a consistent, reproducible composition. Even when it is possible to achieve consistency with respect to the known components of a material isolated from a natural source, it is hard or impossible to ensure that unknown, perhaps as-yet unidentified components that may affect cell properties, are excluded.

Various synthetic materials, chiefly non-degradable or degradable polymers, have been explored for tissue engineering purposes. However, non-degradable materials may constitute a nidus for infection or inflammation, and fragments of some degradable polymers can trigger significant and undesirable inflammatory reactions. In addition, many of these materials may not offer an optimal physical and/or chemical environment for culture of cardiovascular system cells (see, e.g., Vacanti and Langer 1999).

In summary, none of the tissue engineering substrates presently available is fully satisfactory from all points of view. Thus there exists a need for improved tissue culture substrates and scaffolds for use in tissue engineering, particularly for cardiac tissue engineering and angiogenesis. In addition, there exists a need for improved cell culture systems, techniques, and compositions for maintaining cardiovascular system cells and precursors of such cells in vitro and for altering and controlling their differentiation. Furthermore, there exists a need for culture systems and compositions for cell culture that could be implanted into the body, e.g., for tissue engineering purposes.

### SUMMARY OF THE INVENTION

The invention represents the convergence of research in the fields of cardiovascular system cell and tissue biology and biomaterials. The inventors have discovered that a class of peptide hydrogels provide a biologically compatible substrate for growth, differentiation, interaction, and function of a variety of cardiovascular system cells. When cultured on substrates comprising such hydrogels, vascular endothelial cells can survive, migrate, and organize into capillary-like structures.

According to certain embodiments of the invention cardiovascular system cells are cultured on or in 3-dimensional layers or structures comprising self-assembling peptide hydrogels. According to certain embodiments of the invention these layers or structures are comprised of a material that may or may not be crosslinked, has nanometer-scale pores (e.g., 50-200 nanometer diameter), is transparent, and/or is moldable. These features contrast with the crosslinked, micron scale, opaque, and/or rigid nature of various other culture materials. According to certain embodiments of the invention the cells are encapsulated in self-assembling peptide hydrogels either immediately after harvesting or after one or more passages in culture.

Generally, the invention provides methods of culturing cardiovascular system cells comprising culturing the cells on or in a three-dimensional nanoscale environment structure (the term “nanoscale environment structure” is herein used to denote a layer or scaffold with filament diameters in the range of 5-20 nm and inter-fiber spacing in the range of 50-500 nm). According to certain embodiments of the invention the nanoscale environment structure comprises a protein or peptide hydrogel. The hydrogel may be a self-assembling peptide hydrogel as described herein. According to certain embodiments of the invention the peptides comprise amphiphilic peptides, wherein the peptides comprise substantially equal proportions of hydrophilic and hydrophobic amino acids, are complementary and structurally compatible, and are capable of self-assembling into a macroscopic structure or network. According to certain embodiments of the invention the nanoscale environment structure comprises nanofibers. The nanofibers may be comprised of self-assembling peptides capable of forming beta-sheets, e.g., any of the peptides described herein. Beta-sheet filaments may subsequently form higher level structures or networks comprising the three-dimensional gel.

The invention further provides nanoscale environment structures with endothelial cells cultured thereon or within. The nanoscale environment structures may be prepared according to the methods described herein or variations thereof. According to certain embodiments of the invention the nanoscale environment structure comprises a protein or peptide hydrogel. The hydrogel may be a self-assembling peptide hydrogel as described herein. According to certain embodiments of the invention the peptides comprise amphiphilic peptides, wherein the peptides comprise substantially equal proportions of hydrophilic and hydrophobic amino acids, are complementary and structurally compatible, and are capable of self-assembling into a beta-sheet structure, e.g., a filamentous beta-sheet structure, the filaments of which form a macroscopic three-dimensional network. In certain embodiments of the invention the nanoscale environment comprises or consists of an artificial material. According to certain preferred embodiments of the invention, the artificial material comprises or consists of a material not naturally found in the body. Artificial material also encompasses certain materials obtained by isolating and processing substances produced by a living source. However, a material that remains substantially intact and substantially retains the structure in which it is naturally found within the body of an organism is not considered an artificial material. Any of a variety of artificial materials may be used. According to certain embodiments of the invention the nanoscale environment structure comprises nanofibers. The nanofibers may be comprised of self-assembling peptides, e.g., any of the peptides described herein. In certain embodiments of the invention the self-assembling peptides described herein are approximately 5 nm in length and approximately 1 nm in diameter and undergo self-assembly in beta-sheets to form nanofibers (e.g., fibers having a diameter of approximately 10-20 nm). In certain embodiments of the invention the peptides undergo self-assembly to form nanofibers that are highly hydrated (e.g., up to 99.5-99.9%% (1-5 mg/ml) water). Because the hydrogel has such an extremely high water content, cells can freely migrate and form intercellular contacts. Such an environment also permits diffusion of small molecules including proteins and signaling molecules. In addition, certain of the hydrogels have a low elastic modulus. While not wishing to be bound by any theory, the inventors suggest that the low elastic modulus may facilitate cell-cell communication.

According to certain embodiments of the invention the cells comprise isolated cardiovascular system cells, e.g., cells that are not in their natural environment within the body of a subject. For example, the cells may comprise cells that have been removed from a subject. Such cells may have been cultured (e.g., according to conventional cell or tissue culture techniques) following removal prior to culture on or within the nanoscale environment structure. The cells may comprise a cell line. According to certain embodiments of the invention the cells comprise or consist of progenitor cells or stem cells that have the capacity to differentiate into cardiovascular system cells of one or more cell type.

The invention provides methods of treating an individual comprising (i) identifying an individual in need of treatment; and (ii) administering a nanoscale environment structure comprising cardiovascular system cells to the individual. The nanoscale environment structure may be any of the nanoscale environment structures described above. According to certain embodiments of the invention the cells comprise vascular endothelial cells. According to certain embodiments of the invention the cells comprise vascular endothelial cells and a second cell type. In certain embodiments of the invention the second cell type comprises cardiomyocytes.

The invention provides a composition comprising: a macroscopic structure comprising amphiphilic peptides, wherein the peptides comprise substantially equal proportions of hydrophobic and hydrophilic amino acids, are complementary and structurally compatible, and are capable of self-assembling into a beta-sheet macroscopic structure or network; and cardiovascular system cells. In certain embodiments of the invention the peptides assemble into beta-sheet filaments which further assemble to form a macroscopic structure or network. In certain embodiments of the invention the peptide is RAD16-I (Acetyl-RADARADARADARADA-CNH2). According to certain embodiments of the invention the peptides are dissolved in a solution substantially free of electrolytes at a concentration of 0.5% weight/volume prior to self-assembly, or wherein the final concentration of the peptides following self-assembly is between 1 and 10 mg/ml inclusive. The cardiovascular system cells can comprise vascular endothelial cells, angioblasts, cardiac myoblasts, cardiac myocytes, and/or vascular smooth muscle cells. The cells may also comprise fibroblasts and/or smooth muscle cells that are not necessarily of cardiovascular origin. The cells may also comprise embryonic, fetal, or adult stem cells, e.g., stem cells that are able to or can be induced to differentiate into any of the preceding cell types.

In another aspect, the invention provides a method of culturing cells comprising (i) providing vascular endothelial cells; (ii) contacting a plurality of the vascular endothelial cells with a cell culture material comprising amphiphilic peptides, wherein the peptides comprise substantially equal proportions of hydrophilic and hydrophobic amino acids, are complementary and structurally compatible, and are capable of self-assembling into a beta-sheet macroscopic structure, under conditions selected to induce reprogramming in a plurality of the cells. According to certain embodiments of the invention the contacting comprises placing the vascular endothelial cells on the surface of the material, while according to other embodiments the contacting comprises encapsulating the vascular endothelial cells in the material, e.g., by suspending the cells in the peptide solution prior to formation of a gel. The conditions may include presence of a cell culture medium and, optionally, one or more growth factors.

In another aspect, the invention provides a cell composition produced in accordance with any of the methods described above.

In another aspect, the invention provides a pre-vascularized scaffold that can be used as a three-dimensional substrate for cell culture and tissue or organ formation. In particular, the pre-vascularized scaffold can be used for cardiomyocyte culture and cardiac tissue formation.

In another aspect, the invention provides various compositions and methods for treating a subject in need of treatment for a condition, disease, injury, etc. One such method comprises administering, to a subject, a composition prepared according to any of the methods described above. One method comprises administering a composition comprising a cell-containing peptide solution that has not yet formed a gel, and allowing gel formation to occur in vivo.

According to certain embodiments of the invention some or all of the cells are derived from the subject to which they are to be administered. According to certain embodiments of the invention the cells are removed from the macroscopic structure prior to administering them to the subject. The cells may be maintained in tissue culture after removal prior to their administration.

In another aspect, the invention provides a culture kit comprising (i) amphiphilic peptides, wherein the peptides comprise substantially equal proportions of hydrophilic and hydrophobic amino acids, are complementary and structurally compatible, and are capable of self-assembling into a beta-sheet macroscopic structure; (ii) instructions for initiating self-assembly of the peptides into a macroscopic structure; and (iii) at least one of the following items: a population of cardiovascular system cells, cell or tissue culture medium, a predetermined amount of a growth factor, a predetermined amount of an electrolyte, instructions for encapsulating cells within a peptide hydrogel structure and for other uses of the system, a vessel in which the encapsulation may be performed, a liquid in which the peptide can be dissolved, an electrolyte for initiating peptide self-assembly, and one or more reprogramming agents.

The present invention refers to various patents, patent applications, books, and publications in the scientific literature. The contents of all such items are incorporated herein by reference in their entirety. In addition, except as otherwise indicated, the present invention may employ standard cell culture techniques and media and standard molecular biological and immunological protocols such as are found in reference works such as Freshney, R. I., Culture of Animal Cells: A Manual of Basic Technique, 4th ed., John Wiley & Sons, New York, 2000; Ausubel et al., Current Protocols in Molecular Biology, John Wiley & Sons, NewYork, 2000; Harlow, E., Lane, E., and Harlow, E., (eds.) Using Antibodies: A Laboratory Manual, Cold Spring Harbor Laboratory Press, Cold Spring Harbor, 1998.

### BRIEF DESCRIPTION OF THE DRAWINGS

FIG. 1 shows a photograph of an endothelial cell cluster formed by human endothelial cells cultured on a peptide hydrogel structure. FIG. 1A (left) shows staining for von Willebrand factor. FIG. 1B (right) shows staining for DAPI. to identify cell nuclei, demonstrating formation of a cell cluster.

FIG. 2 is a micrograph showing formation of capillary-like structures by 24 hours after seeding on the peptide gel. FIG. 2A shows staining of the actin cytoskeleton. FIG. 2B shows costaining of actin (red) and DAPI staining of cell nuclei (blue) at 24 hours in culture. FIG. 2C (actin staining) is a lower magnification view than FIG. 2A, demonstrating the formation of cell clusters at 24 hours in culture. FIG. 2D (propidium iodide staining) shows the formation of sprouts and inter-cluster connections as well as cell migration into the gel at day 12 in culture.

FIGS. 3A and 3C show BrdU staining (dark brown) of cells cultured on gel and control, respectively, at day 8. FIG. 3E shows results of a TUNEL assay indicating lack of apoptosis when endothelial cells are cultured on peptide gel. FIGS. 3B, 3D, and 3F show DAPI staining of the same regions as shown in FIGS. 3A, 3C, and 3E.

FIG. 4A is a plot of a correlation function.

FIG. 4B is a plot of a correlation analysis for endothelial cells 2 hours after seeding on a peptide gel structure, showing short-range correlation (lack of cell organization).

FIG. 4C is a plot of a correlation analysis for endothelial cells 24 hours after seeding on a peptide gel structure, showing long-range correlation (cell clusters and capillary-like structures).

FIG. 5 is a photograph showing expression of connexin 43 in neonatal rat myocytes co-cultured with human microvascular endothelial cells in three dimensional peptide gel culture. FIG. 5A shows immunostaining for connexin 43. FIG. 5B shows staining for DAPI. (20× magnification).

FIG. 6 shows formation of a capillary-like structure 24 hours after vascular endothelial cell seeding on a peptide gel structure. The scale is the same as in FIG. 2C.

FIGS. 7A and 7B show micrographs of hematoxylin and eosin (7A, 20× magnification) and Masson's trichrome (7B, 100× magnification) stained vascular endothelial cells cultured on peptide gel structures for 10 days.

FIGS. 8A (10× magnification) and 8B (100× magnification) show hematoxylin and eosin stained micrographs of vascular endothelial cells cultured on peptide gel structures for 18 days.

FIG. 9A (20× magnification) shows rat neonatal cardiomyocytes cultured on a peptide gel structure without endothelial cells for 2 days. FIG. 9B (10× magnification) shows rat neonatal cardiomyocytes cultured on an endothelial cell network formed by culturing endothelial cells on peptide gel structure 2 days after seeding.

FIGS. 10A (10× magnification) and 10B (10× magnification) show cardiomyocytes cultured on peptide gel alone or on endothelial cell structures formed on a peptide gel at 18 days after cardiomyocyte seeding.

FIG. 11 is a photomicrograph (10× magnification) showing hematoxylin and eosin stained myocardial tissue into which peptide gel was injected.

### DETAILED DESCRIPTION OF CERTAIN EMBODIMENTS

I. Methods and Compositions of the Invention

The development of new biological materials, particularly biologically compatible materials that serve as permissive substrates for cell growth, differentiation, and biological function has broad implications for advancing medical technology and for understanding basic biological characteristics of cells. The present invention relates to materials and techniques for culturing cardiovascular system cells and stem cells capable of giving rise to cardiovascular system cells, including methods for controlling and altering their properties and influencing their differentiation. The inventors have previously described a class of biomaterials that are made through self-assembly of ionic or polar self-complementary peptides (See, e.g., Zhang, S., et al., Proc. Natl. Acad. Sci. USA, 90, 3334-3338, 1993; Zhang, S., et al., Biomaterials, 16, 1385-1393, 1995; U.S. Pat. Nos. 5,955,343 and 5,670,483). These materials are hydrogels, which in certain embodiments contain approximately 99% or greater water content. They self-assemble into membranes or three-dimensional structures upon exposure to a sufficient concentration of ions to form a stable macroscopic porous matrix composed of orderly interwoven filaments approximately 10-20 nm in diameter with pore size on the order of 50-100 nm in linear dimension. An important characteristic of these materials is that their properties and mechanical strength can be controlled through manipulation of peptide parameters (León et al., 1998; Caplan et al., 2000, Caplan et al., 2001, Zhang et al., 2002). For example, it has been shown that the stiffness of the gel increases strongly with peptide concentration (Leon et al., 1998). The sequences, characteristics, and properties of the peptides and the structures formed by them upon self-assembly are further discussed in the next section.

The inventors have shown that these peptide structures are able to support cell attachment, viability, and growth when cells are cultured on the surface of the structure. In addition, the structures are able to serve as substrates for neurite outgrowth and synapse formation when neurons are grown on their surface (Holmes, T., et al., Proc. Natl. Acad. Sci., 97(12), 2000). In addition, inventors have shown that it is possible to encapsulate cells within the peptide hydrogels, thus placing the cells in a three-dimensional arrangement within the peptide structure (also referred to as a scaffold), and that the cells maintain viability and function when so encapsulated (see pending U.S. patent application Ser. No. 09/778,200, filed Feb. 6, 2001, Entitled “Peptide Scaffold Encapsulation Of Tissue Cells And Uses Thereof” and Provisional Patent Application Ser. No. 60/305,379, entitled “Liver Cellular Reprogramming in Peptide Hydrogel and Uses Thereof”). Inventors showed, for example, that chondrocytes encapsulated within peptide structures are able to synthesize extracellular matrix components.

The inventors have now extended the above findings to encompass the use of peptide hydrogels as culture systems to promote the survival, growth, proliferation, differentiation, migration, and organization of vascular endothelial cells. As described in further detail in the Examples, when cultured on a 3-dimensional peptide hydrogel structure, vascular endothelial cells migrate on and into the gel and organize into capillary-like structures, which exhibit sprouting and formation of inter-cluster connections. Vascular endothelial cells cultured on self-assembling peptide hydrogels thus form a vascular endothelial cell network matrix. The self-assembling peptide hydrogel provides a substrate that triggers a self-sustaining angiogenic process.

Angiogenesis is critical in many normal and pathological processes and has been the subject of intensive research efforts during the past two decades (Carmeliet, 2000, Han & Liu, 1999). These studies resulted in considerable progress in understanding the molecular mechanisms of capillary network formation and remodeling (Carmeliet 2000, Cross & Claesson-Welsh, 2001, Han & Liu, 1999, Ferrara, 2001). Angiogenesis is a complex multi-step process that includes degradation of the extracellular matrix, migration and differentiation of endothelial cells and formation of new lumen structures, which later become capillaries (Carmeliet, 2000). This process is regulated by a tight balance of a variety of factors, including several growth factor families and hypoxia (Cross & Claesson-Welsh, 2001, Ferrara, 2001). Vascular endothelial growth factor (VEGF) family members and angiopoietins, Ang1 and Ang2, are considered to be the critical endothelium-specific factors in vascular development (Ferrara 2001, Gale & Yancopoulos, 1999). VEGF acts on endothelial cells (EC) through three EC-specific receptors, receptor tyrosine kinases VEGFR-1 (Flt-1), VEGFR-2 (Flk-1 or KDR) and VEGFR-3 (Flt-4) and induces endothelial cell proliferation, differentiation and sprouting. Similar to VEGF, angiopoietins Ang1 and Ang2 act on the endothelial cells via EC-specific receptors, Tie1 and Tie2 (Gale & Yancopoulos, 1999). Experimental data suggest that binding of Ang1 to Tie2 plays a stabilizing role for already formed vessels and promotes interactions between endothelial cells and surrounding support cells (Gale & Yancopoulos, 1999). Also, Ang1 was shown to promote EC sprouting in vitro (Koblizek et al., 1998). In contrast, Ang2 binding to Tie2 blocks the stabilizing action of Ang1 resulting in more plastic and unstable vessels (Gale & Yancopoulos, 1999, Maisonpierre et al., 1997). Increasing understanding of the mechanisms that govern angiogenesis opens new opportunities for manipulating this process and designing new pro- and anti-angiogenic treatments (Carmeleit and Rakesh, 2000).

The peptide gel environment offers a number of important features for three-dimensional culture of vascular endothelial cells and use of the resulting vascular endothelial cell network matrix as a scaffold for tissue engineering. In particular, vascular endothelial cells were able to survive, proliferate, differentiate, and organize without the addition of externally supplied angiogenic factors, e.g., angiogenic factors in amounts exceeding what would normally be found in standard tissue culture media, in contrast to results obtained with other three-dimensional substrates used for studies of angiogenesis in vitro, including collagen or fibrin gels or Matrigel (Akeson et al., 2001; van Hinsbergh et al. 2001; Bayless et al, 2000; Ilan et al., 1998, Satake et al. 1998). Furthermore, the inventors found that vascular endothelial cells cultured on three-dimensional peptide hydrogels did not undergo apoptosis, in contrast to previously reported three-dimensional culture systems (Kuzuya et al., 1999; Papapetropoulos et al, 1999; Pollman et al., 1999; Ilan et al., 1998; Satake et al., 1998; Goto et al., 1993). Thus the peptide gels appear to exert a protective or anti-apoptotic effect in comparison with other three-dimensional substrates. In addition, minimum degradation of the peptide gel occurred during more than 3 weeks in culture, also in contrast with previously reported culture systems such as fibrin and collagen gels (Montesano et al, 1987; Collen et al., 1998)

Vascular endothelial cell network matrices formed on and/or in peptide hydrogels have a variety of uses. For example, the vascular endothelial cell network matrices may be used as substrates or scaffolds for the growth of other cell types. In other words, the vascular endothelial cell network matrices can serve as a pre-vascularized scaffold to support the growth of other cells, e.g., for tissue engineering applications. As described in the Examples, the inventors have shown that the vascular endothelial cell network matrices promotes cardiac myocyte survival, organization, and spontaneous coherent contraction. These results indicate that the vascular endothelial cell network matrices are promising substrates for the development of functional blood vessels and/or cardiac tissues in vitro. Development of functional blood vessels may involve, in addition to growth of endothelial cells, addition of cells such as smooth muscle cells and/or fibroblasts, which may not necessarily be of cardiovascular system origin. These cells may be added together with the endothelial cells, prior to formation of the endothelial cell networks. Alternatively, they may be added at any time after the endothelial cell network has formed. Formation of cardiac tissue involves the addition of cardiomyocytes or cells capable of differentiating into cardiac myocytes.

Tissues developed in vitro using the endothelial cell network structures may be used therapeutically, e.g., as functional blood vessels or cardiac tissue grafts. For example, they may be implanted into a subject suffering from circulatory system disease or dysfunction in order to enhance the subject's circulatory system. They may be implanted into a subject in order to cardiac damage or dysfunction, in order to supplement or enhance myocardial function.

In addition, the vascular endothelial cell network matrices may be used to support the growth and proliferation of other cell types. The vascular endothelial cell network matrices may serve as a pre-vascularized scaffold, providing a blood supply to support the growth of a wide variety of tissues and/or organs in vitro. These tissues and/or organs may then be implanted into a subject suffering from tissue or organ dysfunction or damage.

In an alternative approach, the pre-vascularized scaffolds may be used as a culture system to support the growth, proliferation, differentiation, etc., of other cell types, which may then be isolated from the scaffold. The isolated endothelial cells may be used therapeutically.

Inventors continue to explore the influence of the peptide structures on angiogenic phenomena, e.g., the formation and development of capillary-like structures and on the ability of vascular endothelial cell network matrices to support the growth and functional organization, etc., of other cell types such as cardiac myocytes. While not wishing to be bound by any theory, inventors propose a number of possibilities that may be systematically explored and parameters that may be varied in order to refine and expand upon the discoveries and inventions described herein. For example, the peptide sequence, length, and concentration may be varied, which may in turn affect the stiffness, oxygen tension, fraction of cell surface in contact with the gel, and/or growth or differentiation factor gradients within the structure.

Formation of the peptide gel and its mechanical properties are influenced by several factors. Thus, mechanical strength of the gel has been shown to depend on the peptide length, with the intermediate length (12 amino acids) being the stiffest (compared to 8 and 16 amino acids) (Caplan et al., 2000, Caplan, et al., 2001). In addition to peptide length, the material properties of the gel also depend on the peptide concentration, with stiffness of the gel increasing with peptide concentration (Leon et al., 1998). Matrix density and material properties of the peptide gel may be varied to affect cell attachment and migration. Motifs such as the RAD motif, similar to RGD motifs, may be incorporated into the peptide gel, which may enhance cell-matrix interaction and biological function. All such improvements and refinements of the peptide environment structure are within the scope of the invention. In addition, the invention encompasses the addition of cross-linking agents to the peptides, including but not limited to a biotin tag. For example, addition of a biotin tag would allow further strengthening of the gel by subsequent addition of avidin.

The sections below address various aspects of the invention including peptide structures, cells that may be used in the practice of the invention, methods of encapsulating cells within peptide structures of the invention, and inventive culture techniques that may be employed in the context of cells cultured in or on peptide structures, in order to achieve various effects on cell division and phenotype. Methods for monitoring the effects of the peptide structures and culture conditions on cell division and phenotype are also presented. Among the embodiments of the invention are (i) an in vitro culture system for studying angiogenesis; (ii) an in vitro culture system for controlling and manipulating angiogenesis, from which cells can be removed and then either maintained in vitro or administered to a subject; (iii) a vascular endothelial cell network matrix (also referred to as a pre-vascularized scaffold) that may be administered to a subject or used to culture other cells, e.g., cardiomyocytes; (iv) a vascular endothelial cell network matrix further comprising cardiac myocytes cultured thereon, which may be administered to a subject; (v) a vascular endothelial cell network matrix further comprising another cell type, which may be administered to a subject; (vi) a vascular endothelial cell network matrix further comprising cardiac myocytes, which may be administered to a subject. Various other embodiments of the invention such as cell culture kits, methods of culturing cells, and methods of treatment are also described.

II. Peptide Structures and Methods of Culturing and Encapsulating Cells

Inventors have discovered a class of certain peptides consisting of alternating hydrophilic and hydrophobic amino acids that are capable of self-assembling to form an exceedingly stable beta-sheet macroscopic structure in the presence of electrolytes, such as monovalent cations. The peptides are complementary and structurally compatible. These peptides and their properties are described in U.S. Pat. Nos. 5,955,343 and 5,670,483 and in co-pending U.S. patent application Ser. No. 09/778,200, filed Feb. 6, 2001, entitled “Peptide Scaffold Encapsulation Of Tissue Cells And Uses Thereof” and Provisional Patent Application 60/305,379, entitled “Liver Cellular Reprogramming in Peptide Hydrogel and Uses Thereof”.

The following definitions are of use in understanding the peptides and peptide structures formed from them.

The term “nanoscale” generally refers to structures having dimensions that may most conveniently be expressed in terms of nanometers. For example, the term “nanoscale structure” may refer to a structure having dimensions of approximately 500 nm or less, approximately 100 nm or less, approximately 50 nm or less, approximately 20-50 nm, approximately 10-20 nm, approximately 5-10=n, approximately 1-5 nm, approximately 1 nm, or between 0.1 and 1 nm. “Approximately” here means that the measurement may deviate by 10% from the numeral given, and the ranges listed are assumed to include both endpoints. The relevant dimensions may be, e.g., length, width, depth, breadth, height, radius, diameter, circumference, or an approximation of any of the foregoing in the case of structures that do not have a regular two or three-dimensional shape such as a sphere, cylinder, cube, etc. Any other relevant dimensions may also be used to determine whether a structure is a nanoscale structure, depending on the shape of the structure. One of ordinary skill in the art will recognize that one or more dimensions of a nanoscale structure need not be in the nanometer range. For example, the length of such structures may run into the micron range or longer. However, generally most dimensions are in the nanometer range.

As used herein, the term “nanofiber” refers to a fiber having a diameter of nanoscale dimensions. Typically a nanoscale fiber has a diameter of 500 nm or less. According to certain embodiments of the invention a nanofiber has a diameter of less than 100 nm. According to certain other embodiments of the invention a nanofiber has a diameter of less than 50 nm. According to certain other embodiments of the invention a nanofiber has a diameter of less than 20 nm. According to certain other embodiments of the invention a nanofiber has a diameter of between 10 and 20 nm. According to certain other embodiments of the invention a nanofiber has a diameter of between 5 and 10 nm. According to certain other embodiments of the invention a nanofiber has a diameter of less than 5 nm.

The term “nanoscale environment scaffold” refers to a scaffold comprising nanofibers. According to certain embodiments of the invention at least 50% of the fibers comprising the scaffold are nanofibers. According to certain embodiments of the invention at least 75% of the fibers comprising the scaffold are nanofibers. According to certain embodiments of the invention at least 90% of the fibers comprising the scaffold are nanofibers. According to certain embodiments of the invention at least 95% of the fibers comprising the scaffold are nanofibers. According to certain embodiments of the invention at least 99% of the fibers comprising the scaffold are nanofibers. Of course the scaffold may also comprise non-fiber constituents, e.g., water, ions, growth and/or differentiation-inducing agents such as growth factors, therapeutic agents, or other compounds, which may be in solution in the scaffold and/or bound to the scaffold.

The term “microscale” generally refers to structures having dimensions that may most conveniently be expressed in terms of micrometers. For example, the term “microscale structure” may refer to a structure having dimensions of approximately 500 μm or less, approximately 100 μm or less, approximately 50 μm or less, approximately 20-50 μm, approximately 10-20 μm, approximately 5-10 μm, approximately 1-5 μm, approximately 1 μm, or between 0.1 and 1 μm. One of ordinary skill in the art will recognize that the length of such structures may run into the millimeters, but that most dimensions are in the micrometer range.

As used herein, the term “microfiber” refers to a fiber having a diameter of microoscale dimensions. Typically a microscale fiber has a diameter of 500 μm or less, a diameter of less than 100 μm, a diameter of less than 50 μm, a diameter of less than 20 μm, a diameter of between 10 and 20 μm, or a diameter of between 5 and 10 μm.

By “complementary” is meant having the capability of forming ionic or hydrogen bonding interactions between hydrophilic residues from adjacent peptides in the scaffold. Each hydrophilic residue in a peptide either hydrogen bonds or ionically pairs with a hydrophilic residue on an adjacent peptide or is exposed to solvent.

By “structurally compatible” is meant capable of maintaining a sufficiently constant intrapeptide distance to allow structure formation. In certain embodiments of the invention the variation in the intrapeptide distance is less than 4, 3, 2, or 1 angstroms. It is also contemplated that larger variations in the intrapeptide distance may not prevent structure formation if sufficient stabilizing forces are present. This distance may be calculated based on molecular modeling or based on a simplified procedure that has been previously reported (U.S. Pat. No. 5,670,483). In this method, the intrapeptide distance is calculated by taking the sum of the number of unbranched atoms on the side-chains of each amino acid in a pair. For example, the intrapeptide distance for a lysine-glutamic acid ionic pair is 5+4=9 atoms, and the distance for a glutamine-glutamine hydrogen bonding pair is 4+4=8 atoms. Using a conversion factor of 3 angstroms per atom, the variation in the intrapeptide distance of peptides having lysine-glutamic acid pairs and glutamine-glutamine pairs (e.g., 9 versus 8 atoms) is 3 angstroms.

In the case of a peptide scaffold encapsulating cells, by “substantially uniformly distributed” is meant that immediately after scaffold formation at least 50, 60, 70, 80, 90, or 100% of the cells encapsulated by the scaffold are separated from each other by distances that vary by less than 500, 100, 50, 20, 10, or 1 μM.

By “iso-osmotic solute” is meant a non-ionizing compound dissolved in an aqueous solution.

By “solution that is substantially free of electrolytes” is meant a solution to which no electrolytes have been added or in which the concentration of electrolytes is less than 0.01 or 0.001 mM.

Self-assembly is initiated by the addition of an ionic solute to a peptide solution or by a change in pH (Caplan, et al., 2000; Caplan, et al., 2002). For example, NaCl at a concentration of between 5 mM and 5 M induces the assembly of macroscopic structures within a few minutes. Lower concentrations of NaCl may also induce assembly but at a slower rate. The side-chains of the peptides in the structure partition into two faces, a polar face with charged ionic side chains and a nonpolar face with alanines or other hydrophobic groups. These ionic side chains are self-complementary to one another in that the positively charged and negatively charged amino acid residues can form complementary ionic pairs. These peptides are therefore called ionic, self-complementary peptides, or Type I self-assembling peptides. If the ionic residues alternate with one positively and one negatively charged residue (−+−+−+−+), the peptides are described as “modulus I;” if the ionic residues alternate with two positively and two negatively charged residues (−++−++), the peptides are described as “modulus II.”

Many modulus I and II self-complementary peptides with identical compositions and length such as EAK16, KAE16, RAD16, RAE16, and KAD16 have been analyzed previously (Table 1). Modulus IV ionic self-complementary peptides containing 16 amino acids; such as EAK16-IV, KAE16-IV, DAR16-IV and RAD16-IV; has also been studied. If the charged residues in these self-assembling peptides are substituted (i.e., the positive charged lysines are replaced by positively charged arginines and the negatively charged glutamates are replaced by negatively charged aspartates), there are essentially no significant effects on the self-assembly process. However, if the positively charged residues, lysine and arginine are replaced by negatively charged residues, aspartate and glutamate, the peptides can no longer undergo self-assembly to form macroscopic structures; however, they can still form a beta-sheet structure in the presence of salt. Other hydrophilic residues, such as asparagine and glutamine, that form hydrogen bonds may be incorporated into the peptides instead of or in addition to charged residues. If the alanines in the peptides are changed to more hydrophobic residues, such as leucine, isoleucine, phenylalanine or tyrosine, these peptides have a greater tendency to self-assemble and form peptide matrices with enhanced strength. Some peptides that have similar compositions and lengths as these aforementioned peptides form alpha-helices and random-coils rather than beta-sheets and do not form macroscopic structures. Thus, in addition to self-complementarity, other factors are likely to be important for the formation of macroscopic structures, such as the peptide length, the degree of intermolecular interaction, and the ability to form staggered arrays.

Self-assembled nanoscale structures can be formed with varying degrees of stiffness or elasticity. While not wishing to be bound by any theory, low elasticity may be an important factor in allowing the cells in the scaffold to communicate and thereby promote organization and/or differentiation. These peptide scaffolds typically have a low elastic modulus, in the range of 1-10 kPa as measured in a standard cone-plate rheometer. Such low values permit scaffold deformation as a result of cell contraction, and this deformation may provide the means for cell-cell communication. Scaffold stiffness can be controlled by a variety of means including changes in peptide sequence, changes in peptide concentration, and changes in peptide length (Caplan, et al., 2000; Caplan, et al., 2002). Other methods for increasing stiffness can also be used, such as by attaching a biotin molecule to the amino- or carboxy-terminus of the peptides or between the amino- and carboxy-termini, which may then be cross-linked.

TABLE 1
Representative Self-Assembling Peptides

| | | |
|---|---|---|
| Name | Sequence (n-->c) | Modulus |

| | | |
|---|---|---|
| RADA16-I | n-RADARADARADARADA-c | I |
| RGDA16-I | n-RADARGDARADARGDA-c | I |
| RADA8-I | n-RADARADA-c | I |
| RAD16-II | n-RARADADARARADADA-c | II |
| RAD8-II | n-RARADADA-c | II |
| EAKA16-I | n-AEAKAEAKAEAKAEAK-c | I |
| EAKA8-I | n-AEAKAEAK-c | I |
| RAEA16-I | n-RAEARAEARAEARAEA-c | I |
| RAEA8-I | n-RAEARAEA-c | I |
| KADA16-I | n-KADAKADAKADAKADA-c | I |
| KADA8-I | n-KADAKADA-c | I |
| EAH16-II | n-AEAEAHAHAEAEAHAH-c | II |
| EAH8-II | n-AEAEAHAH-c | II |
| EFK16-II | n-FEFEFKFKFEFEFKFK-c | II |
| EFK8-II | n-FEFKFEFK-c | I |
| ELK16-II | n-LELELKLKLELELKLK-c | II |
| ELK8-II | n-LELELKLK-c | II |
| EAK16-II | n-AEAEAKAKAEAEAKAK-c | II |
| EAK12 | n-AEAEAEAEAKAK-c | IV/II |
| EAK8-II | n-AEAEAKAK-c | II |
| KAE16-IV | n-KAKAKAKAEAEAEAEA-c | IV |
| EAK16-IV | n-AEAEAEAEAKAKAKAK-c | IV |
| RAD16-IV | n-RARARARADADADADA-c | IV |
| DAR16-IV | n-ADADADADARARARAR-c | IV |
| DAR16-IV* | n-DADADADARARARARA-c | IV |
| DAR32-IV | n-(ADADADADARARARAR)-c | IV |
| EHK16 | n-HEHEHKHKHEHEHKHK-c | N/A |
| EHK8-I | n-HEHEHKHK-c | N/A |
| VE20* | n-VEVEVEVEVEVEVEVEVEVE-c | N/A |
| RF20* | n-RFRFRFRFRFRFRFRFRFRF-c | N/A |

N/A denotes not applicable
*These peptides form a β-sheet when incubated in a solution containing NaCl, however they have not been observed to self-assemble to form macroscopic structures.

The list presented in Table 1 is representative rather than exclusive. Other self-assembling peptides may be generated, e.g., by changing the amino acid sequence of any self-assembling peptide by a single amino acid residue or by multiple amino acid residues. Additionally, the incorporation of specific cell recognition ligands, such as RGD and RAD, into the peptide structure may promote the proliferation of the encapsulated cells. In vivo these ligands may also attract cells from outside a structure to the structure, where they may invade it or otherwise interact with encapsulated cells. To increase the mechanical strength of the structures, cysteines may be incorporated into the peptides to allow the formation of disulfide bonds, or residues with aromatic rings may be incorporated and cross-linked by exposure to UV light. The in vivo half-life of the structures may also be modulated by the incorporation of protease cleavage sites into the structure, allowing it to be enzymatically degraded. Combinations of any of the above alterations may also be made to the same peptide structure. Formation of cross-links by adding biotin to the peptides and then cross-linking by addition of avidin may also be used and may be a preferable approach. This has been demonstrated in actin gels to increase the elastic modulus by 2 orders of magnitude.

The peptides may include L-amino acids, D-amino acids, natural amino acids, non-natural amino acids, or a combination thereof. If L-amino acids are present in the structure degradation of the structure produces amino acids that may be reused by the host tissue. It is also contemplated that the peptides may be covalently linked to a compound, such as a chemoattractant or a therapeutically active compound. The peptides may be chemically synthesized or purified from natural or recombinant sources, and the amino- and carboxy-termini of the peptides may be protected or not protected. The peptide structure may be formed from one or more distinct molecular species of peptides which are complementary and structurally compatible with each other. Peptides containing mismatched pairs, such as the repulsive pairing of two similarly charged residues from adjacent peptides, may also form structures if the disruptive force is dominated by stabilizing interactions between the peptides. Peptide structures are also referred to herein as peptide hydrogel structures, peptide gel structures, or hydrogel structures.

Peptides capable of being cross-linked may be synthesized using standard f-moc chemistry and purified using high pressure liquid chromatography. The formation of a peptide structure may be initiated by the addition of electrolytes as described herein. Hydrophobic residues with aromatic side chains may be cross-linked by exposure to UV irradiation. The extent of the cross-linking may be precisely controlled by the predetermined length of exposure to UV light and the predetermined peptide concentration. The extent of cross-linking may be determined by light scattering, gel filtration, or scanning electron microscopy using standard methods. Furthermore, the extent of cross-linking may also be examined by HPLC or mass spectrometry analysis of the structure after digestion with a protease, such as matrix metalloproteases. Material strength may be determined before and after cross-linking.

In certain embodiments of the invention peptides forming the macroscopic structure contain between 8 and 200 amino acids, 8 to 36 amino acids, or 8 to 16 amino acids, inclusive. In certain embodiments of the invention the concentration of the peptides is between 1 and 10 mg/ml or between 4 and 8 mg/ml, inclusive.

Aggrecan processing sites may be added to the amino- or carboxy-terminus of the peptides or between the amino-and carboxy-termini. Likewise, other matrix metalloproteases (MMPs) cleavage sites, such as those for collagenases, may be introduced in the same manner. Peptide structures formed from these peptides, alone or in combination with peptides capable of being cross-linked, may be exposed to various protease for various lengths of time and at various protease and scaffold concentrations. The rate of degradation may be determined by HPLC, mass spectrometry, or NMR analysis of the digested peptides released into the supernatant at various time points. Alternatively, if radiolabeled peptides are used, the amount of radiolabeled peptides released into the supernatant may be measured by scintillation counting. Cross-linking and cleavage studies are described further in pending U.S. patent application Ser. No. 09/778,200, filed Feb. 6, 2001, Entitled “Peptide Scaffold Encapsulation Of Tissue Cells And Uses Thereof”.

If desired, the beta-sheet secondary structure of the assembled peptides may be confirmed using standard circular dichroism (CD) to detect an absorbance minimum at approximately 218 nm and a maximum at approximately 195 nm. In addition, peptide structures may be characterized using various biophysical and optical instrumentation, such as dynamic light scattering, Fourier transform infrared (FTIR), atomic force microscopy (ATM), scanning electron microscopy (SEM), and transmission electron microscopy (TEM). Additionally, filament and pore size, fiber diameter, length, elasticity, and volume fraction may be determined using quantitative image analysis of scanning and transmission electron microscopy. The structures may also be examined using several standard mechanical testing techniques to measure the extent of swelling, the effect of pH and electrolyte concentration on structure formation, the level of hydration under various conditions, and the tensile or compressive strength.

Peptide structures may be generated in a variety of shapes and geometries by forming the structure within an appropriately shaped mold (see FIG. 12). Where the peptide structure or scaffold is to be implanted into the body, the shape may be selected based upon the intended implantation site.

The versatile peptide hydrogels may be used in a variety of ways for culturing cells and tissues. Cells and tissues can be cultured on the surface of a hydrogel structure. Under such conditions cells can migrate into the 3-dimensional structure of the hydrogel. While not wishing to be bound by any theory, inventors suggest that such an environment more closely mimics the natural cellular environment than culture on a rigid 2-dimensional substrate. In addition, cells can be encapsulated within the hydrogel. To encapsulate cells within a peptide structure, peptides and living cells may be incubated in an aqueous solution having an iso-osmotic solute (i.e., a solute at an appropriate concentration to support cell viability), under conditions that do not allow the peptides to substantially self-assemble. In certain embodiments of the invention the solution contains less than 10, 5, 1, or 0.1 mM electrolyte or is substantially free of electrolyte. Sufficient electrolyte is added to the solution to initiate self-assembly of the peptides into a beta-sheet macroscopic structure, whereby the cells are encapsulated by the formation of the macroscopic structure. The encapsulated cells are present in the macroscopic structure in a three-dimensional arrangement. In certain embodiments of the invention the concentration of the added electrolyte is at least 5, 10, 20, or 50 mM. Suitable electrolytes include, but are not limited to, Li⁺, Na⁺, K⁺, and Cs⁺. In some embodiments, the concentration of the iso-osmotic solute is at least 50, 150, or 300 mM. In other embodiments, the concentration of the iso-osmotic solute is contained in one of the following ranges 200 to 250 mM, 250 to 270 mM, 270 to 300 mM, 300 to 400 mM, 400 to 500 mM, 500 to 600 mM, 600 to 700 mM, 700 to 800 mM, or 800 to 900 mM, inclusive. Suitable iso-osmotic solutes include, but are not limited to, carbohydrates, such as sucrose, mannitol, etc. Other iso-osmotic solutes. preferably non-toxic to cells at the concentration used, may be employed.

In order to form a macroscopic structure of predetermined shape or volume, peptides and, optionally, living cells may be incubated in an aqueous solution having an iso-osmotic solute, under conditions that do not allow the peptides to substantially self-assemble. In certain embodiments of the invention the solution contains less than 10, 5, 1, or 0.1 mM electrolyte or is substantially free of electrolytes. The solution is contained in a pre-shaped mold dimensioned to determine the volume or shape of the macroscopic structure. Sufficient electrolyte is added to the solution to initiate self-assembly of the peptides into a beta-sheet macroscopic structure, whereby the cells, if present, are encapsulated by the formation of the macroscopic structure. Encapsulated cells are present in the structure in a three-dimensional arrangement. The concentration of the added electrolyte may be at least 5, 10, 20, or 50 mM. Suitable electrolytes include Li⁺, Na⁺, K⁺, and Cs⁺. In one embodiment, the concentration of the iso-osmotic solute is at least 50, 150, or 300 mM. In another embodiment, the concentration of the iso-osmotic solute is contained in one of the following ranges 200 to 250 mM, 250 to 270 mM, 270 to 300 mM, 300 to 400 mM, 400 to 500 mM, 500 to 600 mM, 600 to 700 mM, 700 to 800 mM, or 800 to 900 mM, inclusive. Suitable iso-osmotic solutes include, but are not limited to, carbohydrates such as sucrose, etc.

According to various embodiments of the invention cells and tissues may be cultured on the surface of a peptide structure, cells may migrate into a peptide structure, and/or cells may be encapsulated within a peptide structure, in which case the structure may be referred to as a “scaffold”. When cells or tissues are cultured on the surface of a peptide structure, the 3-dimensional nature of the structure assumes less importance, and the structure may be referred to as a “layer”. However, in contrast to the rigid surface of a conventional culture dish, the peptide structure is not rigid and allows cells to migrate into the structure and/or to extend cellular processes into the structure.

The peptide structures may be used for regenerating a tissue, and the invention includes methods for such use. The methods include administering to an animal, such as a mammal (including a human) a macroscopic peptide structure having amphiphilic peptides and cardiovascular system cells and/or their progeny. The cells may be present on the surface of the structure and/or within the structure. The peptides have alternating hydrophobic and hydrophilic amino acids, are complementary and structurally compatible, and self-assemble into a beta-sheet macroscopic structure. In certain embodiments of the invention the cells include vascular endothelial cells. In certain embodiments of the invention the cells include vascular endothelial cells and a second cell type, which maybe cardiac myocytes.

If encapsulated, the cells are preferably present in the macroscopic structure in a three-dimensional arrangement. The density of the cells may be approximately 10⁵/ml, between 5×10⁵/ml and 5×10⁶/ml, inclusive, between 5×10⁴/ml and 5×10⁵/ml, between 5×10⁵/ml and 5×10⁶/ml. Other ranges may also be used. Conditions for culturing should be close to physiological conditions. The pH of the culture medium should be close to physiological pH, preferably between pH 6-8, for example about pH 7 to 7.8, in particular pH 7.4. Physiological temperatures range between about 30° C. to 40° C. Mammalian cells are preferably cultured at temperatures between about 32° C. to about 38° C., e.g., between about 350 C to about 37° C.

Cells may be cultured on or within the peptide structure for any appropriate time, depending upon the cell number and density desired, the proliferation rate of the cells, and the time required for the desired cellular reprogramming to occur. These parameters will vary depending upon the particular cells and purposes for which the invention is to be used. One of ordinary skill in the art will be able to vary these parameters and to observe the effects of doing so, in order to determine the optimal time for maintaining cells in culture on or within the structure. In certain embodiments of the invention the cell are cultured for approximately 3 days, 7 days, 14 days, 21 days, 28 days, 56 days, or 90 days. In certain embodiments of the invention the cells are cultured for between 1 and 3 days inclusive, between 4 and 7 days inclusive, between 8 and 14 days inclusive, between 15 and 21 days inclusive, between 22 and 28 days inclusive, between 29 and 56 days inclusive, or between 57 and 90 days inclusive. Longer or shorter culture periods may also be used.

As mentioned above, peptide structures may incorporate cells in a variety of ways. Cells may be encapsulated within a peptide scaffold. Cells may be present on the surface of a peptide structure, and/or cells may migrate into the peptide structure. A peptide structure incorporating cells may be used to treat a variety of tissue defects and diseases, particularly defects, diseases, disorders, and injuries of the central and/or peripheral nervous system. The peptide hydrogel structure may be implanted into the body, e.g., surgically or using any other type of suitable procedure. Other routes, including oral, percutaneous, intramuscular, intravenous, and subcutaneous may be employed. One of ordinary skill in the art will be able to select an appropriate delivery technique.

The macroscopic structure may assemble prior to administration, but in certain embodiments of the invention the cardiovascular cells and peptides are mixed in vitro and the structure self-assembles after administration and encapsulates the cells in vivo. As described above, in certain embodiments of the invention the administered solution contains less than 10, 5, 1.0, or 0.1 mM electrolyte or is substantially free of electrolyte, and the concentration of the iso-osmotic solute is at least 50, 150, or 300 mM. In other embodiments, the concentration of iso-osmotic solute is contained in one of the following ranges 200 to 250 mM, 250 to 270 mM, 270 to 300 mM, 300 to 400 mM, 400 to 500 mM, 500 to 600 mM, 600 to 700 mM, 700 to 800 mM, or 800 to 900 mM, inclusive. Suitable iso-osmotic solutes include, but are not limited to, carbohydrates, such as sucrose. In certain embodiments of the invention, the macroscopic scaffold structure is enzymatically degradable. In other embodiments, the macroscopic scaffold is cleavable by a metalloprotease, collagenase, or aggrecanase in vivo or in vitro.

In certain embodiments of the invention the macroscopic structure further encapsulates a therapeutically active compound or chemoattractant. Examples of such compounds include synthetic organic molecules, naturally occurring organic molecules, nucleic acid molecules, biosynthetic proteins such as chemokines, or modified naturally occurring proteins.

In still other embodiments of the invention, the macroscopic structure further incorporates an agent that enhances or promotes differentiation, dedifferentiation, or transdifferentiation, e.g., a growth factor, such as vascular endothelial growth factor, granulocyte macrophage colony stimulating factor, angiopoietin 1 or 2, epidermal growth factor, nerve growth factor, transforming growth factor-β, tumor necrosis factor α, platelet-derived growth factor, insulin-like growth factor, acidic fibroblast growth factor, basic fibroblast growth factor, hepatocyte growth factor, brain-derived neurotrophic factor, keratinocyte growth factor, bone morphogenetic protein, or a cartilage-derived growth factor. Additional agents include various integrins, PECAM, MMP, VE-cadh, CXC, COX2, etc. Combinations of growth factors and/or therapeutic agents or chemoattractants may be used. The macroscopic structure may incorporate an agent that induces reentry into the cell cycle. Such agents may be added to the peptide solution or to the electrolyte solution prior to initiation of self-assembly. In this case the concentration of the agent will likely be substantially uniform within the assembled structure. In certain embodiments of the invention the agent is added to media with which the peptide structure is incubated after addition of cells. After addition to the media, a portion of th agent enters the peptide structure, e.g., through diffusion. This process may create a gradient. Cells on or in different regions of the structure may exhibit different responses to the agent depending upon the concentration of the agent at the location of the cell.

Growth factors are typically used at concentrations ranging between about 1 fg/ml to 1 mg/ml. Frequently growth factors are used at concentrations in the low nanomolar range, e.g., 1-10 nM. In certain embodiments of the invention growth factors are used at concentrations that are not typically used in the prior art or that are not typically found in vivo under normal conditions. For example, growth factors may be used at concentrations that are 5 fold greater, 10 fold greater, 20 fold greater, 100 fold greater, etc., than is typically required to produce effects or than typically occurs in vivo. Titration experiments can be performed to determine the optimal concentration of a particular agent, such as a growth factor, depending upon the particular effects desired. Factors may be added in purified form or as components of a complex biological mixture such as serum.

In certain embodiments of the invention the peptides that assemble to form a macroscopic structure have a sequence that includes an adhesion site, growth factor binding site, or sequence that provides targeting to a cell, tissue, organ, organ system, or site within an animal.

Certain compositions and methods of the present invention may be used to ameliorate the effects of disease or degeneration of an organ, to repair an injury to an organ or other body structure or to form an organ or other body structure. Such organs or body structures include, but are not necessarily limited to, brain, nervous tissue, esophagus, fallopian tube, heart, intestines, gallbladder, kidney, liver, lung, ovaries, pancreas, prostate, bladder, spinal cord, spleen, stomach, testes, thymus, thyroid, trachea, ureter, urethra, and uterus. Thus in addition to vascular endothelial cells, the compositions of the present invention may include a variety of other cell types and/or precursors of such cell types.

In certain embodiments of the invention vascular endothelial cells and/or their progeny that have proliferated and/or organized into capillary-like structures within a peptide structure are removed or extracted from the structure. In addition, in certain embodiments of the invention cells and/or their progeny that have been cultured on or in a pre-vascularized scaffold are removed or extracted from the structure. Removal or extraction may be accomplished by any suitable means, including removal with a suction pipette, mechanical disruption of the scaffold, enzymatic degradation of the structure in vitro, etc. In certain embodiments of the invention the method selected results in removal or extraction of approximately 10% of cells, between 10% and 25% of the cells inclusive, between 25% and 50% of the cells inclusive, between 50% and 75% of the cells inclusive, or between 75% and 100% of the cells inclusive. Methods that result in any convenient range may be selected. The method selected may depend upon the purposes for which the cells are to be used, the number of cells required, etc. In certain embodiments of the invention the viability of the removed or extracted cells is approximately 10% of cells, between 10% and 25% inclusive, between 25% and 50% of cells inclusive, between 50 and 75% of cells, inclusive, or between 75% and 100% of cells inclusive. Methods that result in any convenient range may be selected. The method selected may depend upon the purposes for which the cells are to be used, the number of cells required, etc.

The extracted cells may be further cultured in vitro, either on or in a peptide hydrogel structure or in any other culture vessel. The extracted cells may be administered to a subject by any appropriate route, including intravenous, subcutaneous, oral, percutaneous, intramuscular, or surgical. The administered cells may be used to supplement a tissue, organ, or body structure in need of an enhanced blood supply. The administered cells may synthesize or otherwise supply a therapeutic agent. For example, the administered cells may supply a protein, e.g., an enzyme that the individual lacks. The administered cells may be genetically modified and thus used as a means to deliver gene therapy.

The present invention provides a number of advantages related to the repair or replacement of tissues. For example, these methods enable the penetration of living cells and cellular processes into a peptide structure and/or the encapsulation of living cells by a peptide scaffold in a three-dimensional arrangement and in a substantially uniform distribution, which may promote the viability and proliferation of the cells. The cells are present in an architecture that more closely approximates the natural situation of cells in the body than does culture in a traditional plastic culture dish or other two-dimensional substrate. As demonstrated by electron microscopy, the peptide structures comprise a network of nanofibers with intervening spaces rather than a solid matrix. Such a structure may allow cell penetration and cell-cell interaction in a way that more closely resembles the setting of cells within the body than allowed by other culture techniques and materials. The ability of cells to adhere to a substrate may influence cell morphology. (See, e.g., Powers, M. J., Rodriguez, R. E., Griffith, L. G., Cell-substratum adhesion strength as a determinant of hepatocyte aggregate morphology. Biotech. and Bioeng. 53, 415-426, 1997). The peptide structures also have the advantage of not eliciting a detectable immune or inflammatory response in mammals. Further, the peptide structures exhibit no detectable swelling when added to a saline solution. This lack of swelling is probably due to the high water content of the structure (typically >99%). This property reduces the probability of an unregulated expansion of the structure that could lead to adverse physiological effects on neighboring tissues. Moreover, if desired, the in vivo rate of degradation of the structures may be modulated by the incorporation of protease cleavage sites into the peptides.

III. Cells for Use in the Invention

As mentioned above, cardiovascular system cells for use in the invention can comprise vascular endothelial cells, angioblasts, cardiac myoblasts, cardiac myocytes, and/or vascular smooth muscle cells. The cells may also comprise fibroblasts and/or smooth muscle cells that are not necessarily of cardiovascular origin. The cells may comprise embryonic, fetal, or adult stem cells, e.g., stem cells that are able to or can be induced to differentiate into any of the preceding cell types. Cell types that can be cultured on or in a pre-vascularized scaffold include, but are not limited to, bone marrow cells, periosteal cells, perichondrial cells, fibroblasts, skeletal myoblasts or myocytes, neuronal cells, hippocampal cells, epidermal cells, non-vascular endothelial cells or smooth muscle cells, keratinocytes, basal cells, spinous cells, granular cells, embryonic stem cells, lung cells, immune system cells, ovarian cells, pancreatic cells, cervical cells, liver cells, or foreskin cells.

Sources of the cells may also include fetal or adult organisms, particularly mammals or established cell lines. Numerous established cell lines are known in the art, many of which are available through the American Type Culture Collection (http://www.atcc.org), which also provides references describing these cell lines. In discussing cells and cell lines, the phrase “derived from” indicates that a cell is obtained from a particular source, or that the cell is a descendant of a cell obtained from that source. For example, a liver-derived cell is a cell that is obtained from the liver or the progeny or descendant of such a cell. When the term “progeny” is used herein, it refers not only to the immediate products of cell division but also to the products of subsequent cell divisions, i.e., to cells that are descendants of a particular cell. A cell that is derived from a cell line is a member of that cell line or is the progeny or descendant of a cell that is a member of that cell line. A cell derived from an organ, tissue, individual, cell line, etc., may be modified in vitro after it is obtained. Such a cell is still considered to be derived from the original source.

The Examples describe isolation of human and rodent endothelial cells and rodent myocytes. Methods for isolating cells from other organisms are known in the art. Cells harvested from an individual may be used either with or without a period of expansion in culture. Alternately, cells that have been propagated in culture as a stable cell line may be used. In certain embodiments of the invention the cells are autologous while in other embodiments of the invention the cells are allogencic or xenogeneic. When non-autologous cells are used, the cells may be treated in various ways prior to introduction into the body in order to reduce the likelihood or reduce the extent of an immune system response by the subject. Such treatments can include modifying, masking, or eliminating an antigen on the surface of a cell as described, for example, in PCT/US00/20129.

In certain embodiments of the invention cells are harvested from a subject, e.g., a patient, and a clonal cell line is derived from one or more of these cells. Clonal lines may be obtained by limiting dilution plating or single cell sorting. Methods for deriving clonal cell lines are well known in the art and are described for example in Puck, T. T. and Marcus, P. I., J. (1956) Experimental Medicine 103, 653; Nias, A. H. W. and Lajtha, L. G. (1965) “Clone size distribution in the study of inhomogeneity of growth rates in tissue culture” in Cell Culture, C. V. Ramakrishnan, ed. (Dr. W. Junk Publishers, Netherlands), and Leong, P.-M., Thilly, W. G., and Morgenthaler, S. (1985) “Variance estimation in single-cell mutation assays: comparison to experimental observations in human lymphoblasts at 4 gene loci”, Mutat Res., June-July;150(1-2):403-10. Cells from the cell line are used in the practice of the invention. When intended for treatment of a particular patient, cells from a matched donor may be advantageously used. Cells isolated from an individual or maintained as a cell line may be cultured according to any appropriate technique including standard cell culture techniques prior to their use in the practice of the present invention.

It may be desirable to genetically modify the cells prior to their use in the invention. Numerous methods for introducing exogenous genetic material into cells are well known in the art. (See, e.g., PCT/US00/20129). Such methods typically include introducing genetic material such as a nucleic acid molecule (e.g., DNA) into the cell, wherein the nucleic acid molecule encodes a product to be expressed by the cell. The product can be, for example, a reprogramming agent such as a growth factor, a transcription factor which will in turn induce expression of other gene products, etc. In certain embodiments of the invention it may be desirable to introduce a selectable marker into the cells. In certain embodiments of the invention it may be desirable to introduce a gene that encodes a selectable marker (e.g., a gene encoding a protein that confers drug resistance) or a detectable marker (e.g., GFP) under the control of a tissue-specific promoter. Expression of the detectable marker may then be used as a means to determine whether the cell or its progeny has differentiated, dedifferentiated, or transdifferentiated along a particular cell lineage pathway characteristic of that tissue. The marker may also be used as a means of isolating cells that have differentiated, dedifferentiated, or transdifferentiated along a particular pathway, e.g., by using immunological methods, FACS, etc., or such other methods as are well known in the art. Numerous selectable and detectable markers are known in the art. In addition, tissue-specific, organ-specific, and lineage-specific promoters are well known. Genes may be introduced under the control of either a constitutive or an inducible promoter of which many are known in the art.

In certain embodiments of the invention a therapeutically desirable genetic modification may be made. For example, in a case where an individual harbors a mutation in a particular gene it may be desirable to introduce a wild-type copy of the gene into the progenitor cell for gene therapy purposes. In certain embodiments of the invention it may be desired to introduce a gene encoding a particular receptor, e.g., a growth factor receptor, in order to confer or enhance a particular differentiation, dedifferentiation, or transdifferentiation potential by allowing cells to respond to the growth factor.

As described in the Examples, the density at which vascular endothelial cells are seeded is important in determining whether they will form clusters and a vascular endothelial cell network (i.e., capillary-like structures). The inventors discovered that densities ranging between 2-8×10⁴ cells/cm² were optimal for human endothelial cells. These numbers will vary depending upon the size of the cells. In addition, the optimum number of cardiomyocytes or other cells to be cultured on a pre-vascularized scaffold will vary. One of ordinary skill in the art will be able to select appropriate densities of vascular endothelial cells and other cell types depending upon cell size.

The number of cells to be administered for therapeutic purposes, the relative proportion of cells of different phenotypes, and/or the concentration of cells within a peptide structure can be altered as appropriate for the particular condition or injury to be treated.

IV. Environmental Factors and Agents For Use in the Invention

As is well known in the art, numerous environmental factors are likely to play key roles in influencing cell and tissue differentiation and development, including angiogenesis. These may include physical or mechanical factors such as compressive forces, contact with substrate, etc. The extracellular matrix is known to exert profound effects on cell development. In addition, cell-cell contacts may play an important role. In addition, shear stress on the cells within or on the surface of the matrix may be significant, e.g., it may be an important stimulus for angiogenesis.

Cell or tissue differentiation and/or development may be induced by a large number of chemical agents. Among these are specific growth and/or differentiation factors, some of which are mentioned above. Over 2,000 growth factors have been identified, and one of ordinary skill in the art will be able to select and to test these factors appropriately depending upon the cellular properties desired. The growth factors may be provided in a pure form or as components of a more complex biological mixture such as serum (e.g., horse serum, fetal bovine serum, calf serum, etc.). The growth factors may be present within the culture medium of a peptide hydrogel structure on which cells are cultured or into which cells migrate or are encapsulated. Growth factors may be encapsulated within the structure itself. In addition, it is well known in the art that different concentrations of a particular growth factor may exert different effects on target cells. One of ordinary skill in the art will be able to test a range of concentrations and combinations in order to achieve the desired effects.

In addition to growth factors, various other chemical stimuli or conditions may be used in the context of the present invention. Among such stimuli are activators of the phosphatidyl inositol pathway, or other factors that increase levels of inositol trisphosphate and/or intracellular Ca⁺⁺ concentrations, activation of protein kinase C and/or other cellular kinases, etc. The presence of small molecules including small organic molecules or metal ions may also influence cellular reprogramming and may be used in the practice of the invention. In addition, the presence of motifs such as an RAD or RGD motif, or of cell matrix components, may influence formation of the vascular endothelial cell networks and/or development of tissues cultured on the pre-vascularized scaffold.

In addition, oxygen concentration and nutrient availability may influence angiogenesis. For example, hypoxia induces a variety of transcription factors that trigger angiogenesis and arteriogenesis. Metabolic stimuli such as hypoglycemia and low pH also stimulate vessel growth. (See, e.g., Carmeliet, 2000, for discussion.)

V. Monitoring Cell Division and Phenotype and Cardiovascular Tissue Formation

It may be desirable to monitor cell division, cell phenotype, and/or the functional state of the cells. As is well known in the art, there are a number of methods for monitoring cell division and for assessing various aspects of cell behavior and phenotype. In general, any appropriate method may be employed to investigate and assess the effects of culturing cells under the conditions described herein. In addition, the effects of the cells on the overall composition and properties of a cell/hydrogel assembly may be monitored. Such features as protein content, strength, etc., can be examined.

Cell morphology may be assessed by visual examination under a light microscope and/or by other microscopic techniques. Cell viability may be assessed by examining vital dye exclusion (e.g., trypan blue exclusion). Cell division may be observed by light microscopy and is indicated by the presence of mitotic figures. An increase in cell number accompanying division may also be observed, e.g., by counting with a hemacytometer. Morphological changes such as cell rounding may also accompany division. DNA synthesis may be monitored by detecting and/or measuring incorporation of various substances such as radiolabeled nucleotides (e.g., ³[H] thymidine), bromodeoxyuridine (BrdU), etc., into DNA.

Methods for assessing apoptosis are well known in the art and include visual examination, TUNEL, and determination of the level of mRNA or proteins associated with apoptosis, e.g., caspases.

Cell differentiation, dedifferentiation, and transdifferentiation can be assessed based on a number of parameters, including morphology. Cell differentiation, dedifferentiation, and transdifferentiation may also be assessed by detecting and/or measuring the presence of certain polypeptides or polynucleotides known as markers. The latter approach is widely used, and cellular markers characteristic of numerous different cell types have been identified. mRNA and/or protein expression may be detected by techniques well known in the art. For example, mRNA may be detected and quantified using Northern blots, cDNA or oligonucleotide microarray analysis, RT-PCR, etc. Protein expression may be detected and quantified using immunoblotting analysis, immunofluorescence, FACS analysis, ELISA assays, etc. Such immunological techniques may employ monoclonal or polyclonal antibodies that bind to the protein.

The variety of markers is immense, and new markers are routinely being identified. Of particular significance in the context of the present invention are markers that may be used to identify vascular endothelial cells, cardiac myocytes, and functional activity of cardiac myocytes. Von Willebrand factor is currently the most widely recognized marker for vascular endothelial cells. Other markers for vascular endothelial cells include CD31, DC102, CD106, and isolectin B4 (Williams K S, 1995; “Microvascular endothelium from adipose tissue” Endothelial Cell Culture. Roy Bicknell (ed). Cambridge University Press, 1996). The markers of angiogenesis include angiogenesis-related growth factors VEGF, Angiopoictins 1 and 2, and their receptors Flt-1, Flk-1, Tie2 (Ferrara, 2001; Gale and Yancopoulos, 1999). Monoclonal antibodies directly conjugated with fluorescent dyes that bind to Various of these are commercially available, e.g., from Dako, Chemicon, etc.

Formation of functional cardiac tissue may be assessed using a variety of techniques known in the art including molecular, structural, and electrophysiological methods (Papadaki, M., et al., 2001) For example, parameters such as cellularity, conduction velocity, maximum signal amplitude, capture rate, and excititation threshold may be measured. Survival and function of engineered cardiac tissue may be tested in vivo using various model systems for cardiac damage as described, for example, in Li, et al., 1999. Numerous such animal model systems are known in the art.

VI. Additional Embodiments and Equivalents

The invention provides kits that may be used for culturing cardiovascular system cells. The kits comprise a peptide hydrogel of the invention, which may be provided in dry or lyophylized form. The kits may further comprise one or more of the following items: instructions for culturing endothelial cells on or within a peptide hydrogel structure, instructions for encapsulating cells within a peptide hydrogel structure, a vessel in which encapsulation may be performed, a liquid in which the peptide can be dissolved, an electrolyte for initiating peptide self-assembly, medium for tissue culture, cardiovascular system cells, etc. Additional items may also be included.

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain important additional information, exemplification and guidance, which can be adapted to the practice of this invention in its various embodiments and the equivalents thereof.

### EXAMPLES

### Example 1

### Endothelial Cell Behavior in Three-Dimensional Peptide Gel

Materials and Methods

Peptide gel preparation and cell isolation and seeding. Peptides (RAD16-11: AcN-RARADADARARADADA-CNH₂) were obtained from Research Genetics (an Invitrogen Corporation, Huntsville, Ala.). To prepare the three-dimensional gel, 150 μl of peptide solution at a concentration (1% w/v) in distilled water was added to 30 mm diameter Millipore culture inserts (Millicell® Culture Plate Inserts, Millipore, Inc., Bedford, Mass.) which were immediately immersed in an external well of a 6-well plate containing PBS (GibCo) and kept at 4° C. for 30 min. After formation of the gel, inserts were filled with culture medium and left in an incubator (37° C., 5% CO₂) overnight before cell seeding. For controls, the wells of 6-well culture plates were coated either with 1% gelatin or with 2 mg/ml collagen I (Vitrogen). Gelatin was dissolved in distilled water (10 g/liter), autoclaved, poured in the wells and left for 30 min. For collagen coating, 3.125 ml of collagen solution (3.2 mg/ml) was added to 1.875 ml of Medium 199 (Gibco), and pH of the solution was adjusted to 7.0. 1 ml of collagen solution was added to each well of a 6-well culture plate, and the plate was kept in an incubator for 2 hr (37° C., 5% CO₂). Before seeding, gelatin solution, as well as excess collagen solution, were aspirated from the wells, and the culture medium was added.

Endothelial cells were isolated from human fat tissue according to a previously published protocol (Williams, 1995) and cultured on gelatin-coated T-75 flasks up until passage 4-6 using endothelial cell culture medium (Medium 199 (Gibco), 10% FCS, 1% penicillin-streptomycin, 1% L-glutamin, 5% heparin (Sigma), 1% ECGS (BD Biosciences).

Cells were trypsinized and seeded on the surface of the assembled peptide gel or control well at a variety of densities including (1) between 5×10³ cells/cm² and 2×10⁴ cells/cm²; (2) between 2-8×10⁴ cells/cm²; and (3) between 8×10⁴ cells/cm² and 5×10⁵ cells/cm². At the lower densities there was no structure formation, while at the highest densities cells cover the entire surface of the gel, and there is no place for them to migrate. For animal cells (such as rat or mice), which are much smaller, the densities needed for capillary formation are higher (approximately, 5 times or more). Cultures were maintained in an incubator (37° C., 5% CO₂), and the media was changed every 3 days.

Immunofluorescence staining. Cell phenotype was evaluated by immunofluorescence staining for von Willebrand factor using rabbit anti-human-von Willebrand factor antibody (DAKO Carpinteria, Calif.) The following protocol was used for all immunofluorescent staining:

Cells on the gel were fixed with 2% formaldehyde for 2 hr at room temperature. Gels were washed with PBS (3×20 min) and permeabilized in 0.1% TritonX-100 in PBS for 1 hr at room temperature. Cultures were incubated in blocking buffer (10% serum in PBS with 0.1% TritonX-100 and 10% DMSO, either FCS or serum from species corresponding to species in which secondary antibodies were raised was used). Cultures were incubated with primary antibodies (1 microgram per milliliter) for 6-8 hours on a shaking table at 4° C. Then cultures were washed with blocking buffer (3×3 hr) and incubated with secondary antibodies for 6-8 hr on a shaking table at 4° C. Finally, cultures were washed with blocking buffer (3×3 hr) and observed under fluorescence microscope.

Detection of apoptosis: Cells undergoing apoptosis were identified using the TUNEL assay (Roche) following the manufacturer's protocol.

Quantitation of cell migration and formation of capillary-like structures. In order to characterize capillary structure formation a new method was developed, which is based on correlation analysis of the images of developing structures. Cell nuclei were stained with DAPI, resulting in greater image intensity at positions corresponding to cell nuclei. In the analysis, a correlation function F(a,b) is calculated for a gray scale image of the cell network at a given time (e.g., FIG. 2A), using the equation below, where (x,y)<

$$F ( a , b ) = \frac{< ( I ( x , y ) - {I}_{ave} ) ( I ( x + a , y + b ) - {I}_{ave} ) >}{{σ}^{2}}$$

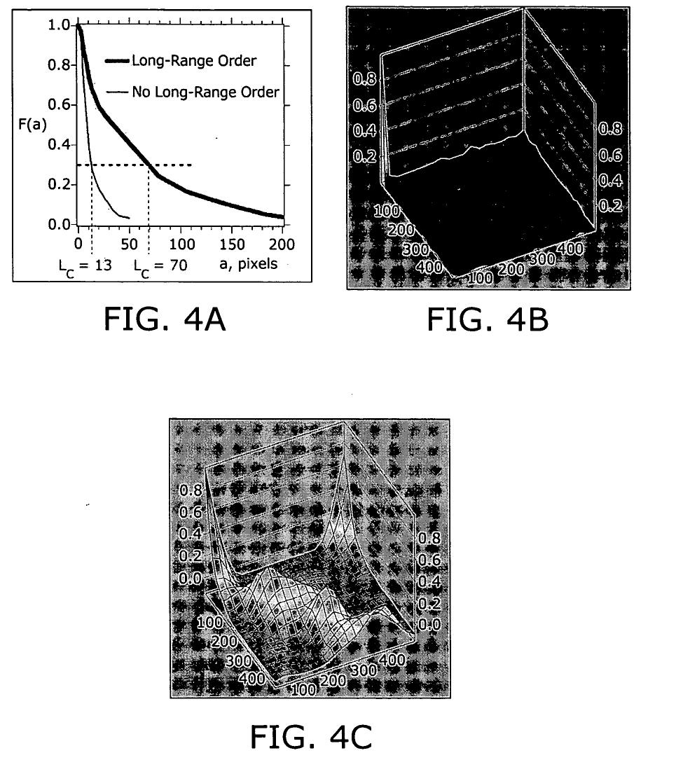

represent pixel coordinates on the original image, (a,b) represent correlation distances in pixels along the x and y directions, I(x,y) is image intensity at (x,y), “°” denotes averagings over (x,y), Iₐᵥₑ is average image intensity, and σ² is standard deviation for I(x,y). The function F(a, b) is then used to obtain correlation length L_{c}, as shown in FIG. 4 (top left). The correlation length, L_{c}, describes the extent of cell spatial organization and characteristic size of forming structures. By determination of L_{c} as a function of time for cells cultured on or in a given substrate, it is then possible to quantitatively characterize the differences in cell migration and structure formation between various substrates.

Results

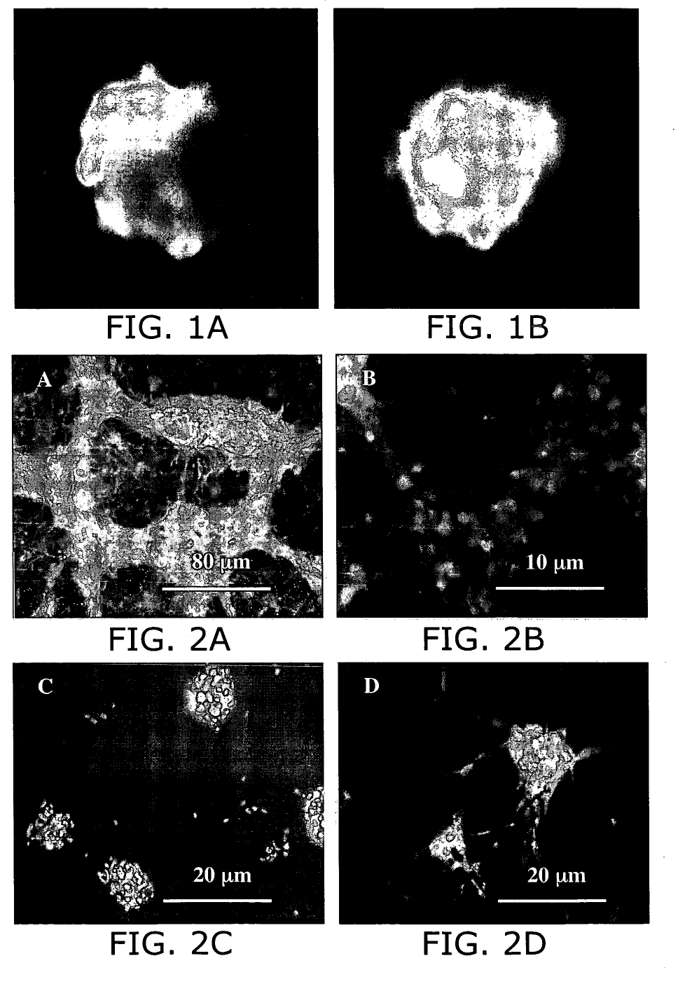

Endothelial cell migration, proliferation, differentiation, survival, andformation of capillary-like structures. When cells were seeded on the peptide gel at densities ranging from 2-8×10⁴ cells/cm², cells attached and started to migrate toward each other within 2 hours of seeding, resulting in formation of clusters. The earliest time points for staining was 2 hours after seeding. Prior to that time cells were observed in microscope. Initial structures were formed by 8 hours, and structure formation was complete by 24 hours, with most cells (more than 90%) assembled into clusters and forming a capillary-like network. FIG. 1B shows staining for DAPI (right) to identify cell nuclei, demonstrating formation of a cell cluster. This staining was done at day 3. FIG. 1A shows staining of the same cell cluster for von Willebrand factor, indicating that the cells retain their endothelial cell phenotype. FIG. 2 is a micrograph showing formation of capillary-like structures by 24 hours after seeding on the peptide gel. FIG. 2A shows staining of the actin cytoskeleton. FIG. 2B shows costaining of actin (red) and DAPI staining of cell nuclei (blue) at 24 hours in culture. FIG. 2C (actin staining) is a lower magnification view than FIG. 2A, demonstrating the formation of cell clusters at 24 hours in culture. FIG. 2D (propidium iodide staining) shows the formation of sprouts and inter-cluster connections as well as cell migration into the gel at day 12 in culture. Extensive cell migration into the gel was observed by day 5 in culture. Cell migration was observed both using fluorescent staining and by light microscopy (magnification 20× and higher). By focusing at different depths, one can see the cells in clusters at different positions in the gel.

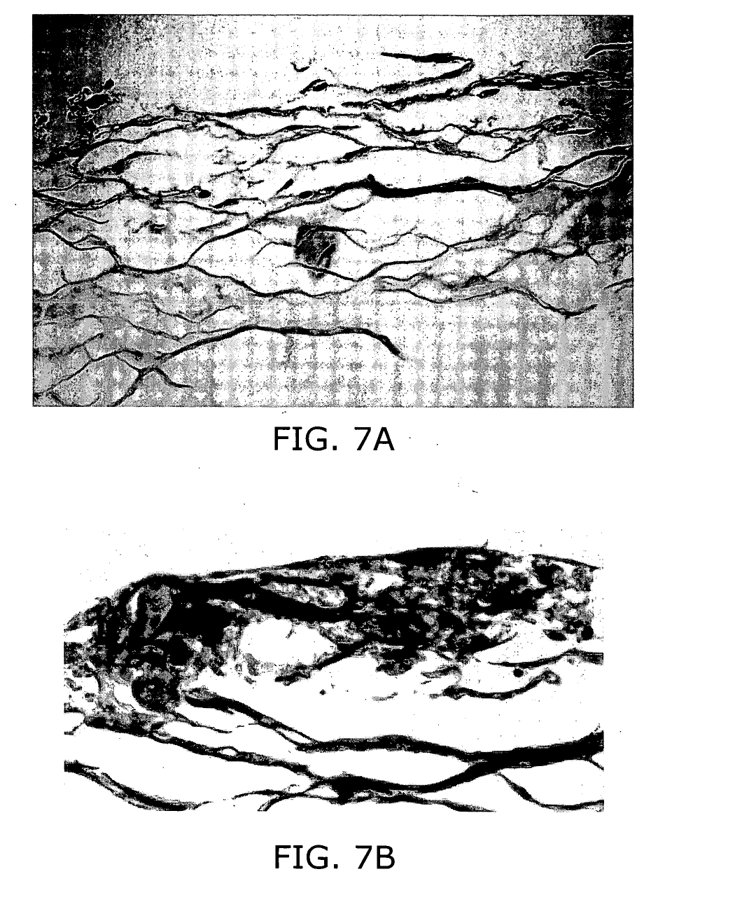

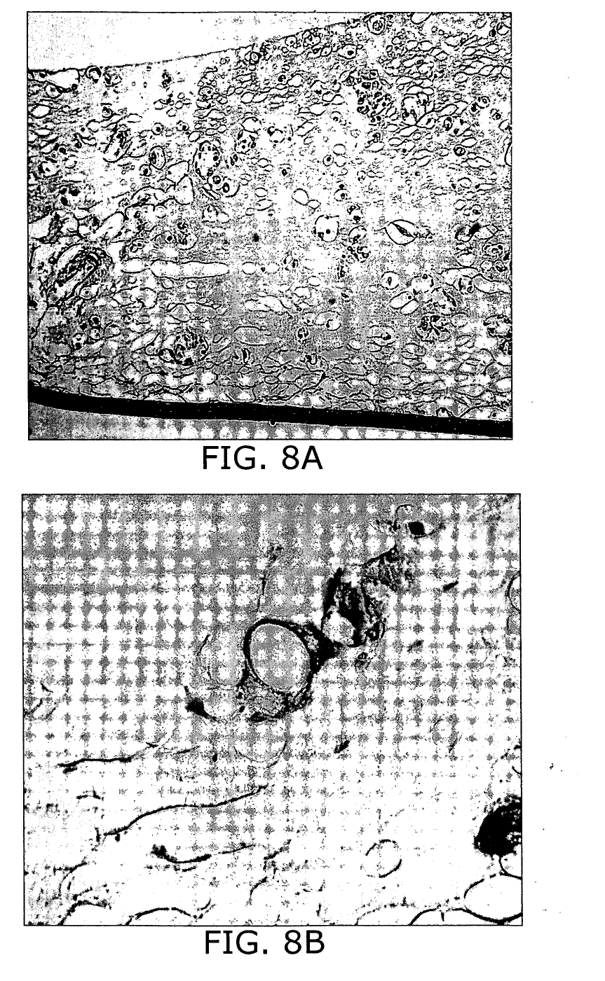

FIG. 6 shows formation of a capillary-like structure 24 hours after endothelial cell seeding on a peptide gel structure. This image strongly suggests sprouting and formation of a lumen. FIGS. 7A and 7B show micrographs of hematoxylin and eosin (7A) and Masson's trichrome (7B) stained endothelial cells cultured on peptide gel structures for 10 days. Small nuclei of cells lining a potential channel are visible in FIG. 7A. A purple endothelial cell curving around and making a lumen can be seen at the upper center of FIG. 7B. FIGS. 8A and 8B show hematoxylin and eosin stained micrographs of endothelial cells cultured on peptide gel structures for 18 days. Numerous cell clusters are evident in FIG. 8A. Note the capillary-like structure (dark signet ring shape) near the middle of FIG. 8B. In these experiments extensive migration of endothelial cells into the peptide gels was observed.

Cell seeding density was found to influence the formation of structures, with densities less than 2×10⁴ cells/cm² resulting in no significant cell migration and structural organization. In contrast, densities between 2-8×10⁴ cells/cm² resulted in formation of cell clusters (FIGS. 1 and 2C), with subsequent sprouting and formation of inter-cluster connections at 12 days in culture (FIG. 2D). Cell densities greater than 1 cells/cm² resulted in formation of large interconnected networks (FIG. 2A-B).

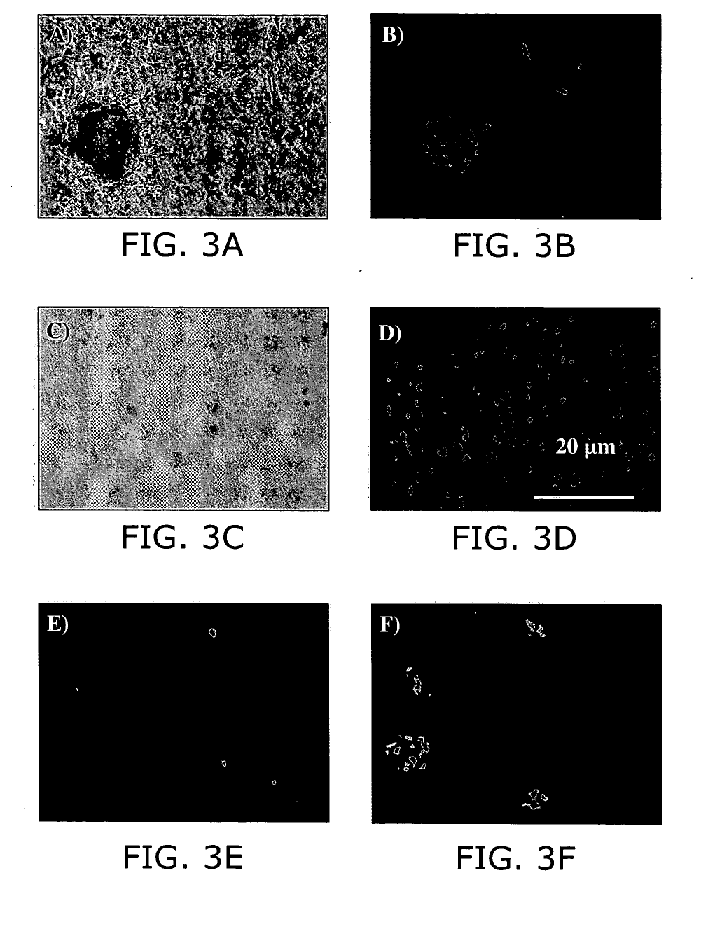

BrdU staining at days 3 and 8 in culture (to identify newly synthesized DNA) was performed to determine whether cells cultured on gels or control substrate were actively proliferating. Cells were seeded at a density of 5×10⁴ cells/cm² on either peptide gel or control. FIGS. 3A and 3B show BrdU staining (dark brown) of cells cultured on gel and control, respectively, at day 8. An actively proliferating cell cluster is visible in the lower left quadrant of FIG. 3A. In contrast, cells grown on control substrate had reached confluence by day 8 and demonstrated very little proliferation. FIGS. 3B and 3D show DAPI staining of the same regions as shown in FIGS. 3A and 3C, respectively, demonstrating the cell cluster formed on the gel and the lack of cell cluster or capillary formation on the control substrate. The active proliferation observed at day 8 when cells were cultured on peptide gels is in contrast to previously reported data for sprouting angiogenesis in collagen I and fibrin gels (Goto et al., 1993, Vailhe et al., 1997, Nehls and Herrmann, 1996) and Matrigel (Kubota et al., 1988).

In contrast to other three-dimensional substrates (Kuzuya et al., 1999, Papapetropoulos et al., 1999, Pollman et al., 1999, Ilan et al., 1998, Satake et al., 1998, Goto et al., 1993), the peptide gel was found to have a protective effect against apoptosis of endothelial cells, with no significant apoptosis detected up to day 8 of culture (FIG. 3, E-F).

Gel degradation in this system was minimal over a three week culture period, with only occasional clefts and gaps observed on the gel surface. This lack of degradation contrasts with the significant proteolysis observed in fibrin and collagen gels (Montesano et al., 1987, Collen et al., 1998).

Correlation analysis was used to quantify the extent of cell organization and structure formation on the surface of the peptide gels as compared with controls and to evaluate cell organization and structure as a function of time. Only short-range correlation (no cell organization) was observed immediately after seeding on peptide gels (or control substrate), resulting in a small L_{c} which corresponds to an average size of the nucleus of about 13 pixels (FIG. 4C). In other words, only pixels within a cell nucleus are significantly correlated in terms of their location, indicating that there is little correlation between the locations of individual cells. For cells cultured on control substrate, the correlation length remained approximately 13 pixels after 24 hours in culture (FIG. 4A). In contrast, there was a long-range correlation for the capillary networks on the gel, with a characteristic correlation length of 70 pixels after 24 hours in culture (FIGS. 4A, 4B). In other words, when cells were grown on the gels the locations of individual cells were significantly correlated, i.e., cells were more likely to be found in clusters or parts of the network than alone.

In summary, these data suggest that the peptide gel supports endothelial cell attachment, migration, proliferation and capillary-like structure formation and survival for a period of at least 3 weeks. (Cultures were maintained for as long as 4-5 weeks, but most data was obtained for the initial 3 week period.) In contrast to other three-dimensional substrates that have been used for studies of angiogenesis in vitro, including collagen or fibrin gels or Matrigel (Akeson et al., 2001, van Hinsbergh et al, 2001, Bayless et al., 2000, Ilan et al., 1998, Satake et al., 1998), this angiogenic response was observed in the absence of externally supplied angiogenic factors, and with no significant signs of apoptosis or proteolytic gel degradation. These results indicate that the self-assembling peptide gel provides a substrate that triggers a self-sustaining angiogenic process in endothelial cell culture.

### Example 2

### Cardiac Myocyte Survival, Organization, and Spontaneous Coherent Contraction on Three-Dimensional Peptide Gel with Endothelial Cell Networks

Materials and Methods

Preparation of endothelial cell network structures. Human endothelial cells were isolated, seeded at a density of 4×10⁴ cells/cm² and cultured on RAD16-II peptide gel structures as described in Example 1 for three weeks to allow formation of endothelial cell networks.

Cardiac myocyte isolation. Neonatal rat cardiac myocytes (CM) were isolated using enzymatic digestion as described in the following protocol:

Mouse Neonatal CM Preparation

Medium preparation: Medium 199, 20% FBS (vol/vol), 100 U/ml penicillin and 100 μg/ml streptomycin.

Digestion solution: HBSS plus100 μg/ml collagenase type II, 1× pancreatin (25 ml).

Isolation of cardiac myocytes:

1. Rinse the neck of neonatal mouse with 70% alcohol.

2. Cut the mouse head with the scissor.

3. Use 1 of the small scissors to open the chest of the mouse, take the heart with forceps.

4. After finishing collect the hearts, put the plate containing the hearts in HBSS into the cell culture hood, cut off the atria and put the ventricles into a new plate containing 15 ml HBSS. Then cut the cardiac ventricles into small pieces using a scissor and transfer the quartered hearts to another plate containing 15 ml HBSS with the Moria spoon.

Cell Digestion:

1. Transfer of quartered hearts to the bottle containing 10 ml collagenase/pancreatin solution, shake slowly in the 37° C. waterbath for 45 minutes.

2. Collect the collagenase/cell suspension into the 50 ml tube. Leave the tube on ice.

3. Add another 10 ml of collagenase/pancreatin solution to non-digested tissue, shake slowly in the 37° C. waterbath for another 20 min and collect the suspension.

4. Centrifuge the cells for 8 min at 200 g, and resuspend the cells in the FBS/Medium 199.

5. Pre-plate the cells for 1 hour (to allow attachment of fibroblasts and endothelial cells). Collect the cells that did not attach, and seed them on the desired substrate.

Cardiac myocyte seeding. Neonatal rat cardiac myocytes were seeded at 4×10⁴ cells/cm² on top of the endothelial cell network structures formed on and in the peptide gel. Cultures were maintained in endothelial cell culture medium (described above).

Medium was changed every 3 days.

Immunofluorescence staining. Formation of gap junctions was evaluated by immunofluorescence staining for connexin 43 using mouse anti-connexin 43 antibody (Chemicon).

Photography and Observation of cells. Contractions were observed using light microscopy (magnifications 20×, 30× and 40×). Movies were recorded at the rate of 25 frames/second.

Results

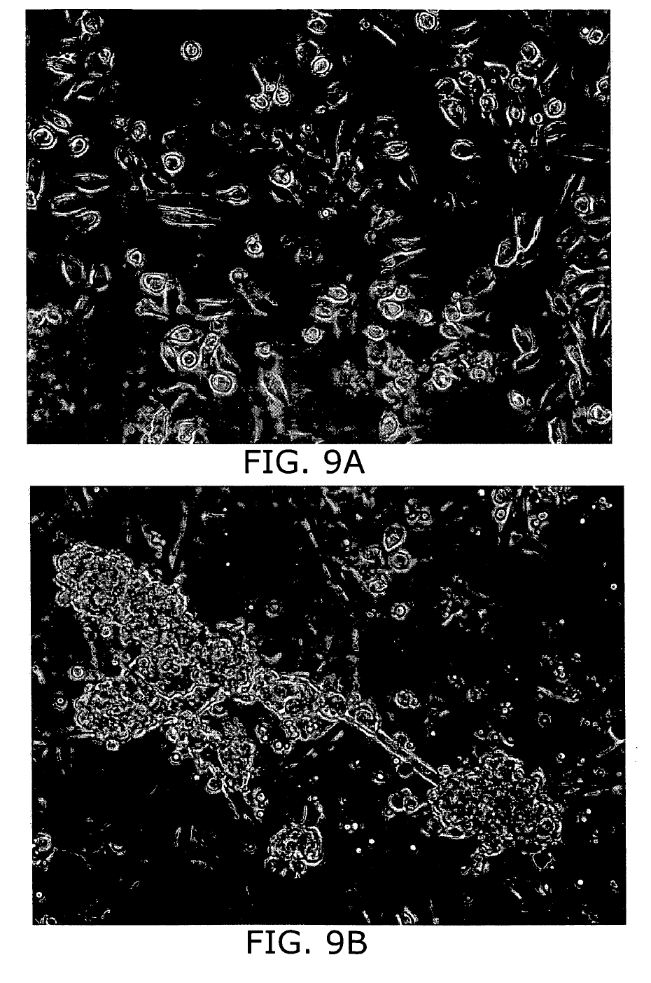

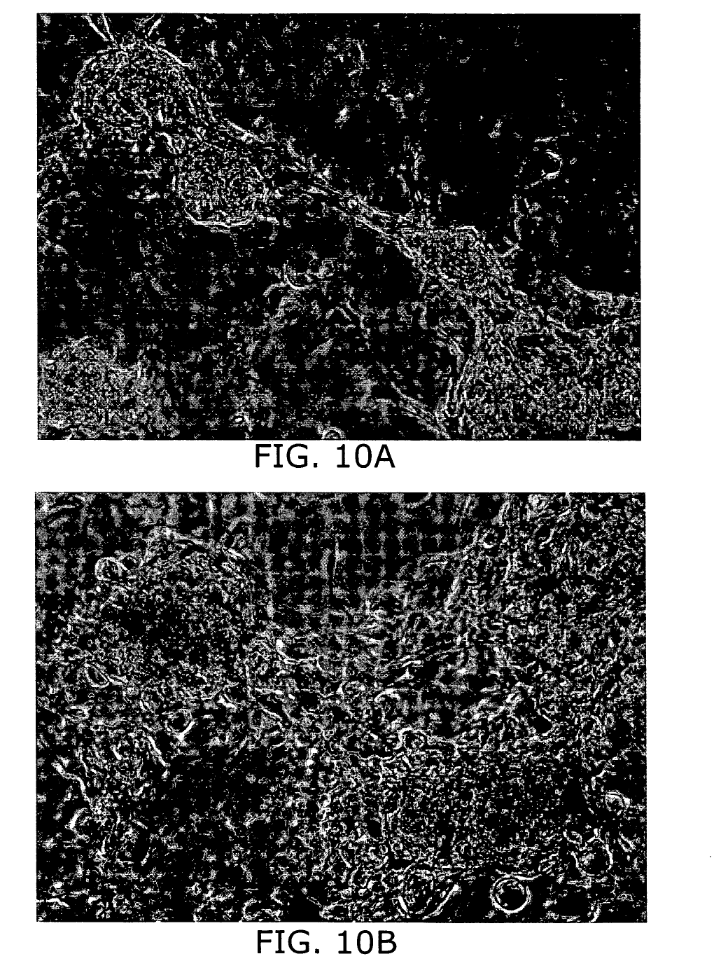

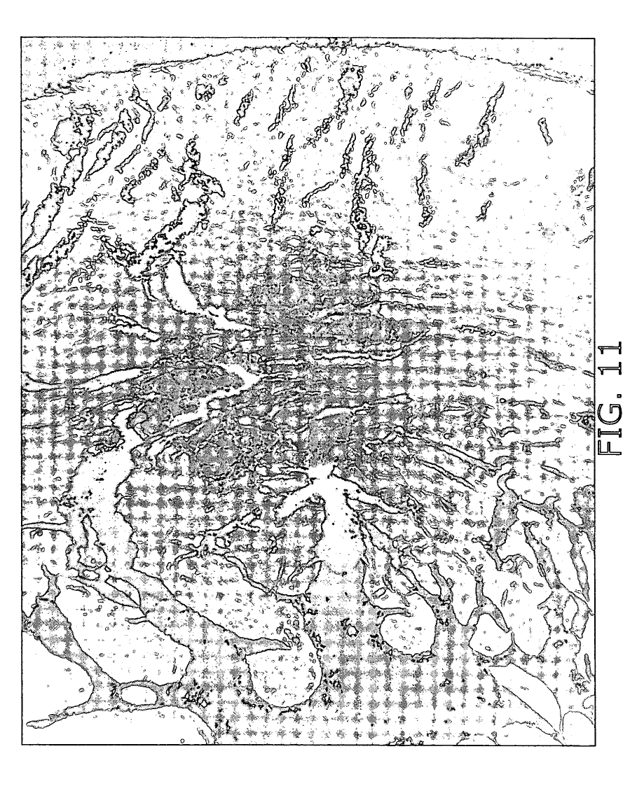

FIG. 9A shows rat neonatal cardiomyocytes cultured on a peptide gel structure without endothelial cells for 2 days. FIG. 9B shows rat neonatal cardiomyocytes cultured on an endothelial cell network formed by culturing endothelial cells on peptide gel structure 2 days after seeding. Cardiomyocytes cultured on the endothelial cell-gel structure aggregated around the endothelial cell clusters whereas cardiomyocytes cultured on the peptide gel alone are more randomly located. FIGS. 10A and 10B show cardiomyocytes cultured on peptide gel alone or on endothelial cell structures formed on a peptide gel at 18 days after cardiomyocyte seeding. At day 2 after seeding, myocytes appeared to attach and migrate into the gel around endothelial cell clusters and exhibited a characteristic beating. By day 4 in culture, spontaneous coordinated cell contractions were observed over a distance of ˜0.5 mm. The synchronized beating continued until 2 weeks in culture.

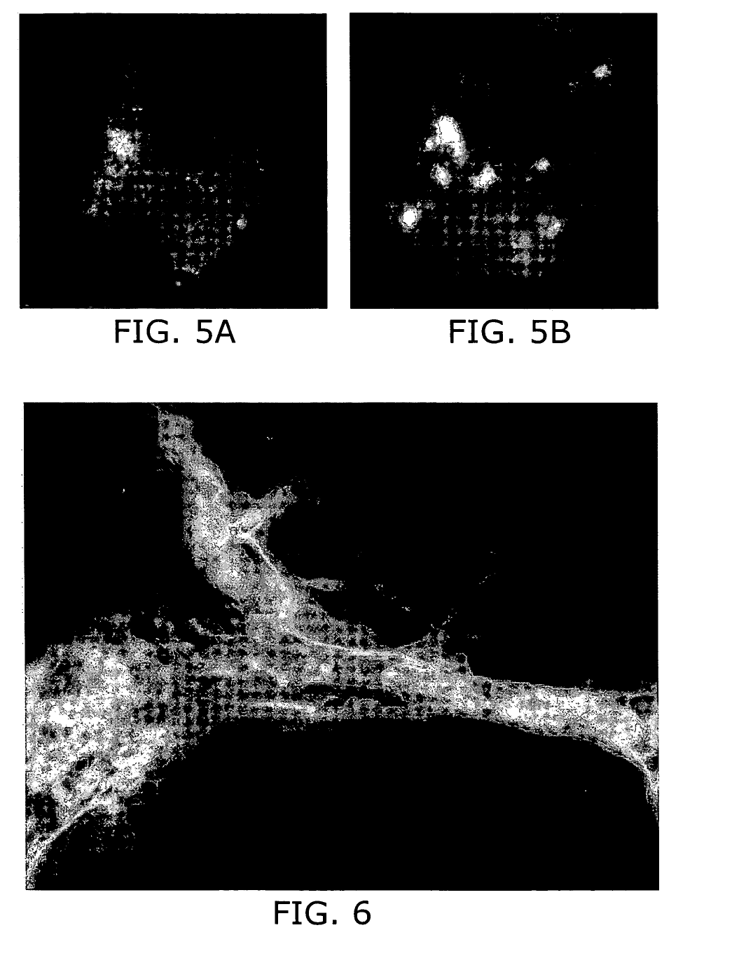

At 3 weeks after seeding, cell cultures were fixed and immunostained for expression of the gap junction marker connexin 43 (van Veen et al, 2001). FIG. 5A shows expression of connexin 43 in cardiomyocytes after 3 weeks of co-culture with endothelial cell networks formed on a peptide gel structure. FIG. 5B shows the same region as FIG. 5A, stained for DAPI to identify endothelial cell and cardiomyocyte nuclei.

These results provide evidence of three-dimensional organization of neonatal cardiomyocytes in clusters adjacent to endothelial cells, with expression of connexin 43, suggesting the formation of functional gap junctions. The presence of synchronized beating further indicates that the cardiomyocytes display attributes of functional myocardial tissue.

### Example 3

### Myocardial Injection of Peptide Gel Structure

Materials and Methods

Peptide Gel Preparation. A 0.6% gel of the RAD 16-II peptide was prepared in syringes 90 minutes before injection and wrapped with plastic to prevent possible evaporation from syringes.

Peptide Injection. C57/BL6 female mice were maintained under standard laboratory conditions. Prior to injection mice (4 months old) were anesthetized. For injection into the myocardium, the chest was opened according to standard techniques, and 20 microliters of peptide gel was injected into the antero-apical region of the left ventricle using a 30 gauge needle while the heart was beating. The chest cavity was closed and mice were returned to their cages. For injection into leg muscle, 20 microliters of the peptide gel were injected into tibialis anterior muscle of the mouse with 30-gauge needle. Twenty-four hours after injection the heart or the leg was harvested, paraffin-embedded, and stained with hematoxylin and eosin according to standard protocols (e.g., Lindsey M L, Gannon J, Aikawa M, Schoen F J, Rabkin E, Lopresti-Morrow L, Crawford J, Black S, Libby P, Mitchell P G, Lee R T, “Selective matrix metalloproteinase inhibition reduces left ventricular remodeling but does not inhibit angiogenesis after myocardial infarction. Circulation. 2002 Feb. 12;105(6):753-8.).

Results

To determine the effects of the peptide gels on living tissue, peptide gel structures were injected into leg muscle or myocardium of living mice. A very slight discoloration was observed during and after injection into myocardial tissue, similar to that of a myocardial infarction. The heart did not stop beating and appeared to be functioning normally, with a constant heart rate after injection. The mice recovered uneventfully from anesthesia and showed no untoward effects.

FIG. 11 is a photomicrograph showing hematoxylin and eosin stained myocardial tissue into which peptide gel was injected. In this figure the endocardium (inside of the left ventricle) is on the left and the epicardium (exterior layer of the heart) is on the right. Cell nuclei are stained purple. The pale pink relatively acellular areas are islands of peptide gel in the heart.

No adverse effects were observed following injection of peptide gel into the leg muscles of over a dozen mice. These results suggest that peptide gel structures are suitable for applications involving introduction of the structures into living muscle tissue.

### Example 4

### Optimization of Peptide Gel Environment for Angiogenesis

Materials and Methods

Peptide gel preparation and endothelial cell isolation and seeding. These are performed as described in Example 1 except that a number of different peptides are used representing a range of peptide lengths and a variety of sequences (e.g., RAD16-I, RAD16-11, KFE12, KLD12—see Table 1). In addition, peptide gel structures are prepared using various different peptide concentrations, e.g., 0.5%, 1%, 1.5%, 2%.

Endothelial cell encapsulation in peptide hydrogel. Endothelial cells are isolated as described in Example 1 and suspended in media. Peptide solutions are prepared by dissolving peptide in deionized, distilled, sterile water at a range of concentrations (e.g., 0.5%, 1%, 1.5%, 2% w/v). Cells are mixed with peptide solution at various final cell concentrations ranging between approximately 104 to 10⁶ cells/ml. The cell/peptide mixture is loaded into multiwell (96-well) plates at 50 μl/well. Immediately after loading, 200 μl of culture medium is added to each well, thereby providing an electrolyte concentration sufficient to allow self-assembly of the gel into a three-dimensional structure. After gel self-assembly, the media is changed three to four times to allow proper equilibration of the cell/hydrogel assembly. The multiwell plates are cultured at 37° C. in a standard incubator containing a humidified chamber equilibrated with 5% CO₂. Cell viability is measured by staining with trypan blue according to standard techniques. (See Kisiday et al. 1999 for further discussion of encapsulation).

Collagen gel preparation and endothelial cell seeding. Endothelial cells are isolated as described in Example 1 and suspended within neutralized collagen solution type I (1.5 mg/ml) and 1 volume of Medium 199 as described in Satake et al., 1998. The cell suspension is then added to culture plates and allowed to gel for 20 min at 37° C., followed by addition of endothelial cell culture medium.

Ribonuclease Protection Assay. Endothelial cells are removed from the peptide or collagen gel by trypsin digestion and centrifugation. For the collagen gel, cultures were incubated with 2 ml/well 0.1% collagenase type I (Worthington Biochemical Corporation) for 10 min, following with addition of 2 ml of culture medium, removal of cell suspension and centrifugation at 400 g for 5 min. Cell pellet was resuspended in PBS and centrifuged again, to remove medium from the suspension. For the peptide gel, gel and cells were removed from the membranes using a cell scraper, pipetted several times to break the gel, centrifuged at 400 g for 5 min, resuspended in PBS and centrifuged again. Cell pellets were then used for RNA extraction. RNA was extracted using a Qiagen kit (RNeasy mini kit 250) according to the manufacturer's instructions. For RPA, the Riboquant system is used (Pharmingen). ³²P-UTP-labeled riboprobes were generated by in vitro transcription. 20 μg of RNA was hybridized with the riboprobes and digested with RNase A following the manufacturer's instructions. A custom made angiogenesis template set (Pharmingen) was used. Samples are separated by SDS-PAGE electrophoresis, and the gel is exposed by radiography.

Quantitation of cell migration and formation of capillary-like structures. Cell migration and capillary structure formation are assessed using correlation analysis as described in Example 1. The method is appropriately modified to analyze endothelial cell structure formation in three dimensions. For some experiments, three dimensional imaging systems are used to assess cell migration and capillary-like structure formation.

Transmission electron microscopy (TEM). Cell cultures in the gel are prepared for TEM by incubating the matrices in 5% glutaraldehyde at 4° C. for 30 minutes, followed by slowly sequential dehydration steps incrementing ethanol volume fraction by 5% for 5 minutes each. The sample is placed in a pressurized liquid CO₂/syphon for 1 hour. The sample is then coated with gold particles, mounted on a grid and examined at 100-2000× magnification (Zhang et al., 1995).

Analysis and interpretation. Analysis of variance (ANOVA) is performed to identify differences in correlation length and mRNA expression between different experimental groups and time points. In general, experiments are repeated with at least triplicate measurements at each time point and at least 3 times with different cell sources since primary cells are used.

The angiogenic response of endothelial cells to the external environment is assessed through quantitation of the time course of capillary formation and evaluation of expression of mRNA encoding various polypeptides associated with angiogenesis including vascular endothelial growth factor (VEGF), angiopoitins Ang1 and Ang2, VEGF receptors Flt-1 and Flk-1, and angiopoietin receptor Tie2. Cell organization and formation of capillary like structures is quantitated at various time points, e.g., 2 hr, 8 hr, 12 hr, 24 hr, 3 days, 1 week and 2 weeks after seeding by determining the correlation length. Staining with hematoxylin and eosin, Massone's trichrome, as well as immunostaining for actin enables visual assessment of endothelial cell cluster formation, sprouting, capillary-like structure formation. The presence of a lumen in the capillary-like structures is assessed visually and by using automated imaging systems including three-dimensional imaging systems. Apoptosis is also assessed using a TUNEL assay (Roche) following manufacturer's protocol. To characterize cell attachment to the matrix and resulting matrix deformation, transmission electron microscopy (TEM) is performed for cell+gel samples at various times, e.g., 2, 8, 24 hours and 3, 14 and 21 days after seeding at 400-2000× magnification.

Varying peptide concentration alters the physical properties of the external environment experienced by the endothelial cells. For example, higher peptide concentrations result in a peptide structure with increased stiffness. Cell-matrix interactions between the endothelial cells and the peptide gel will vary for different peptide structures, which will be detected as differences in the time course of cell migration and capillary structure formation. For example, gels with higher peptide concentration may promote stronger cell attachment and slower migration, compared with less dense gels.

TEM performed at the initial time period (e.g., up to 24 hours) will provide additional information on the extent of cell attachment to the peptide gel structure and possible re-orientation of the peptide filaments in response to cell “pulling” on the gel. TEM performed at later time periods (e.g., days 3, 14 and 21) will be analyzed for signs of peptide matrix degradation and new matrix deposition by the cells. Variations in cell-matrix interactions for peptide structures with different sequences and peptide concentrations may result in differences in endothelial cell proliferation, survival and mRNA expression of angiogenic factors. For example, higher gel concentrations may promote increased proteolytic gel degradation and cell apoptosis, similar to the mechanism reported for endothelial cell cultures in collagen gel (Kuzuya et al., 1999, Satake et al., 1998).

The influence of peptide length, sequence, and concentration as well as the culture method (seeding endothelial cells on formed peptide gel structures or encapsulating endothelial cells in peptide gel structures) on endothelial cell migration, cell-cell interaction, cell survival, capillary structure formation, expression of angiogenic markers, etc., is assessed and compared with these parameters when endothelial cells are cultured on a conventional substrate (collagen). This systematic comparison allows identification of culture parameters that influence angiogenesis in vitro and selection of culture parameters that result in optimum angiogenesis, e.g., for tissue engineering and therapeutic applications.

### Example 5

### Optimization of Endothelial Cell-Gel Structures for Cardiomyoctye and Stem Cell Culture

Materials and Methods

Cardiomyocyte and stem cell isolation. Neonatal mouse or rat cardiomyocytes are isolated as described in Example 2. Adult mouse or rat cardiomyocytes are isolated as described in the following protocol:

Dissection:

1) Sacrifice a young adult mouse by cervical dislocation (or CO₂ inhalation).

2) Soak skin with 70% EtOH.

3) Nick skin above the pubis with scissors and deglove the skin to above the chest.

4) Pin mouse supine on a Styrofoam board on a bench top and spray chest/abdomen with 70% EtOH. Remove any loose fur from gloves or field. Switch to second set of instruments.

5) Cut open anterior abdomen, and take down the anterior diaphragm with sterile scissors.

6) Grasp lobes of the lung and heart and cut free from the mediastinum.

7) Place lobes/heart in 15 mL cold PBS in a 10 cm dish.

8) Repeat Steps 1-7 for a second mouse. Combine lobes from two mice.

C. Tissue Dissociation:

1) Cut off the heart, cut the heart in half and place in 50 mL tube containing 45 mL of cold PBS. Gently agitate.

3) Cut out the lung lobes from any visible bronchi and mediastinal connective tissue, cut each lobe in half. Place in 50-mL conical tube with 45 mL of cold PBS. Gently agitate.

4) Put lung lobes in a dry 10-cm dish and mince finely with scissors for 1 min. (Repeat for heart in a separate dish).

5) Put lung pieces in 25 mL of pre-warmed, 37° C. Collagenase (2 mg/mL). Wash all bits off the plate. (Repeat same for heart).

6) Incubate at 37° C. with gently agitation for 45 min (shaking waterbath).

7) Using a 30 cc syringe attached firmly to the cannula, triturate the suspension 12 times, taking care to avoid frothing.

8) Let chunks settle. Pipette suspension through a 70 um disposable cell strainer (Falcon #35 2350) into a 50 mL conical tube. Wash sieve with 10 mL of medium (Medium 199 with 10% FCS, 1% penicillin/streptomycin, 1% L-glutamin).

9) Spin down cell suspension at 400 g (1300 rpm in GH3.7 rotor), 8 min., 4° C.

10) Resuspend in 2 mL of cold PBS+0.1% BSA and count nucleated cells.

D. First Cell Sorting:

1) Dilute cell suspension to 30×10⁶ cells/mL cell concentration. (Expected approximately 60-80×10⁶ cells from lung prep and 8-15×10⁶ cells from 2 hearts. (Resuspend the heart cells in 1 ml PBS+0.1% BSA).

2) Transfer to a 5-mL polystyrene round-bottom tube. Add 60 uL of Anti-mouse PECAM-1 Dynabeads to 2 mL cell suspension.

3) Incubate on a rotator, RT, 10 min.

4) Add cell suspension to 8 mL of Medium in 15-mL round-bottom polypropylene tube mounted in a magnetic separator (e.g., Dynal MPC-1) and leave for 1-2 min.

5) Remove supernatant. Remove tube from magnetic separator and resuspend beads+cells in 10 mL medium (see above) by vigorous trituration.

7) Mount in magnetic separator for 1-2 min.

8) Repeat Steps 5-7 until the supernatant is clear (usually 5-8 washes).

9) Resuspend in Growth Medium (Medium 199 with 20% FCS, 1% penicillin/streptomycin, 1% L-glutamin, 5% heparin and 1% ECGS) and plate beads+cells in gelatin-coated T75 flasks.

10) The next day, rinse flask three times with isolation media to remove any loosely adherent cells.

E. Second Sort:

1) Feed with Growth Medium every 3 days.

2) Let cells grow until approaching confluence at 5-9 days after plating.

3) Detach cells with Trypsin:EDTA (0.05%:0.5M), add 10 mL of isolation medium to inactivate Trypsin.

4) Transfer cell suspension to a 50 mL tube and spin down at 400 g for 4 min.

5) Remove supernatant and resuspend cell pellet in 2 mL of PBS+0.1% BSA.

6) Add 30 uL ICAM-2 coated SAR Dynabeads. Incubate for 10 min RT.

7) Add 2 mL cell suspension and beads to 8 mL isolation media. Separate on magnet.

8) Wash gently 2× in 10 mL media.

9) After final wash resuspend in 10 mL growth medium and plate in gelatin coated T75.

Peptide gel preparation and endothelial cell isolation and seeding. These are performed as described in Example 4.

Preparation of peptide-coated membrane. Membrane was coated by covering with peptide solution in the absence of salt, allowing the solution to dry, and then repeating the procedure.

Endothelial cell and/or cardiomyocyte encapsulation in peptide hydrogel. These are performed as described for endothelial cells in Example 4. For experiments in which endothelial cells and cardiomyocytes are encapsulated together, a range of concentrations of both cell types is used.

Cardiac myocyte seeding. This is performed as described in Example 2.

Northern blot analysis for connexin 43. Total RNA is isolated using a Qiagen kit (RNeasy mini kit 250) according to the manufacturer's instructions, separated on 1.0% agarose/formaldehyde gel (2 M), transferred to a nylon membrane (Stratagene), and UV cross-linked. The membrane is hybridized with ³²P-CTP-labeled rat connexin 43 cDNA probe (generated by PCR) in QuikHyb solution (Stratagene) at 68° C. for 1 h, washed, and exposed by radiography. Normalization of RNA is performed by hybridizing the membranes with a GAPDH cDNA probe. Connexin 43 and GAPDH mRNA levels are quantitated using Optimas 5.0 densitometry software. RPA and Western blots are performed to further assess expression of connexin 43.

Immunofluorescence staining. Cell phenotype is evaluated by immunofluorescence staining for connexin 43 to identify gap junctions. Cardiomyocyte phenotype is confirmed by staining for two markers: α-sarcomeric actin and α-Sr-1. Antibodies to these markers stain both neonatal and mature cardiomyocytes). Because endothelial cells also express connexin 43 (Carter et al., 1996), staining with α-sarcomeric actin and/or α-Sr-1 antibodies and connexin 43 antibody is used for identification of myocyte gap junctions in co-culture experiments.

Myocyte proliferation is assessed for all experimental groups using double staining with BrdU and α-sarcomeric actin at various times, e.g., days 3, 7, 14 and 21 in culture. Similarly, myocyte apoptosis is characterized using double staining with TUNEL assay and α-sarcomeric actin at various times, e.g., days 3, 7, 14 and 21 in culture.

Transmission electron microscopy (TEM). This is performed as described in Example 4.

Analysis and interpretation. This example compares the ability of various substrates and spatial environments to support cardiomyocyte survival and structural and functional organization into myocardium-like tissue. Cardiomyocytes are either (i) seeded onto the surface of a two-dimensional membrane coated with self-assembling peptide; (ii) seeded onto the surface of an endothelial cell network produced by culturing endothelial cells on a three dimensional peptide hydrogel structure; or cardiomyocytes are encapsulated in a self-assembling peptide hydrogel structure either alone (iii) or in combination (iv) with endothelial cells. Various different peptides and peptide concentrations are used. The ability of various substrates and spatial environments to support cardiomyocyte survival and structural and functional organization into myocardium-like tissue is determined by assessing cardiomyocyte survival, gap junction formation, cardiomyocyte structural organization, matrix production, and evidence of cardiomyocyte functional activity (e.g., coordinated contractions). In particular, the ability of endothelial cell networks produced by culturing endothelial cells on peptide hydrogel structures to enhance cardiomyocyte survival and structural and functional organization into myocardium-like tissue is assessed. In addition, the influence of encapsulation within the three-dimensional peptide environment (with or without endothelial cells) on cardiomyocyte survival and structural and functional organization into myocardium-like tissue is assessed. This work allows identification of culture parameters and environment that result in optimum cardiomyocyte survival and structural and functional organization into myocardium-like tissue, which can be used for various tissue engineering and therapeutic applications.

### REFERENCES

Akeson A L, Brooks S K, Thompson F Y, and Greenberg J M. 2001. In vitro model for developmental progression from vasculogenesis to angiogenesis with a murine endothelial precursor cell line, MFLM-4. Microvasc Res 61:75-86.

Akins R E, Boyce R A, Madonna M L, Schroedl N A, Gonda S R, McLaughlin T A, Hartzell C R. Cardiac organogenesis in vitro: reestablishment of three-dimensional tissue architecture by dissociated neonatal rat ventricular cells. Tissue Eng;5(2):103-18.

Ando J, Tsuboi H, Korenaga R, Takada Y, Toyama-Sorimachi N, Miyasaka M, Kamiya A. Am J Physiol 1994;267(3 Pt 1):C679-87. Shear stress inhibits adhesion of cultured mouse endothelial cells to lymphocytes by downregulating VCAM-1 expression.

Bayless K J, Salazar R, and Davis G E. RGD-Dependent vacuolization and lumen formation observed during endothelial cell morphogenesis in three-dimensional fibrin matrices involves αᵥβ₃ and αᵥβ₁ integrins. Am J Path 2000;156:1673-1683.

Braunwald E, Bristow M R. Congestive heart failure: fifty years of progress. Circulation 2000; 102:IV-14-IV-23.

Bursac N, Papadaki M, Cohen R J, Schoen F J, Eisenberg S R, Carrier R, Vunjak-Novakovic G, Freed L E. Cardiac tissue engineering: cell seeding, cultivation parameters, and tissue construct characterization. Biotechnol Bioeng. 1999;64(5):580-9.

Caplan M, Moore P, Zhang S, Kamm R & Lauffenburger D. Self-assembly of a beta-sheet oligopeptide is governed by relief of electrostatic repulsion relative to van der Waals attraction. Biomacromolecules 2000; 1, 627-631.

Caplan M, Schwartzfarb E, Zhang S, Kamm R, Lauffenburger D. Control of Self-assembling Oligopeptide Matrix Formation Through Systematic Variation of Amino Acid Sequence. Biomaterials 2002;23(1):219-27.

Carmeliet P, Jain R K. Angiogenesis in cancer and other diseases. Nature 2000;407:249-257.

Carmeliet P. Mechanisms of angiogenesis and arteriogenesis. Nat Med 2000;6:389-395.

Carter T D, Chen X Y, Carlile G, Kalapothakis E, Ogden D and Evans W H. Porcine aortic endothelial gap junctions: identification and permeation by caged InsP3. J Cell Sci 1996;109:1765-1773.

Collen A, Koolwijk P, Kroon M E, and van Hinsbergh V W M. Influence of fibrin structure on the formation and maintenance of capillary-like tubules by human microvascular endothelial cells. Angiogenesis 1998.;2:153-165.

Cross M J and Claesson-Welsh L. FGF and VEGF function in angiogenesis: signaling pathways, biological responses and therapeutic inhibition. Trends Pharmacol Sci 2001;22(4):201-207.

Davis G E and Camarillo C W. An a2b1 integrin-dependent pinocytic mechanism involving intracellular vacuole formation and coalescence regulates capillary lumen and tube formation in three-dimensional collagen matrix. Exp Cell Res 1996;224:39-51.

Eschenhagen T, Fink C, Remmers U, Scholz H, Wattchow J, Well J, Zimmermann W, Dohmen H H, Schafer H, Bischopric N, Wakatsuki T, Elson E L. Three-dimensional reconstitution of embryonic cardiomyocytes in a collagen matrix: a new heart muscle model system, FASEB J 1997;11:683-694.

Ferrara N. Role of vascular endothelial growth factor in regulation of physiological angiogenesis. Am J of Physiol (Cell Physiology) 2001;280:C1358-C1366.

Gale N W and Yancopoulos G D. Growth factors acting via endothelial cell-specific receptor tyrosine kinases: VEGFs, Angiopoietins and ephrins in vascular development. Genes Dev 1999; 13:1055-1066.

Goto F, Goto K, Weindel K, and Folkman J. Synergistic effects of vascular endothelial growth factor and basic fibroblast growth factor on the proliferation and cord formation of bovine capillary endothelial cells within collagen gels. Lab Invest 1993;69:508-517.

Griffith L G, Wu B, Cima M J, Powers M J, Chaignaud B, Vacanti J P. In vitro organogenesis of liver tissue. Ann N Y Acad. Sci. 1997;831:382-97.

Han Z C, Liu Y. Angiogenesis: state of the art. Int J Hematol 1999;70(2):68-82.

Holmes T C, de Lacalle S, Su X. Liu G, Rich A, and Zhang S. Extensive neurite outgrowth and active synapse formation on self-assembling peptide scaffolds. PNAS 2000;97:6728-6733.

Holmes T C. Novel peptide-based biomaterial scaffolds for tissue engineering. Trends Biotechnol 2002;20(1):16-21.

Ilan N, Mahooti S, Madri J A. Distinct signal transduction pathways are utilized during the tube formation and survival phases of in vitro angiogenesis. J Cell Sci 1998; 111:3621-31.

Kellar R S, Landeen L K, Shepherd B R, Naughton G K, Ratcliffe A, Williams S K. Scaffold-based three-dimensional human fibroblast culture provides a structural matrix that supports angiogenesis in infarcted heart tissue. Circulation 2001;104(17):2063-8.

Kim E J, Li R K, Weisel R D, Mickle D A, Jia Z Q, Tomita S, Sakai T, Yau T M. Angiogenesis by endothelial cell transplantation. J Thorac Cardiovasc Surg 2001;122(5):963-971.

Kisiday J, Jin M, Hung H, Kurz B, Zhang S, Grodzinsky A. Self-assembling peptide scaffold for cartilage tissue engineering. Trans ORS 2001;26:54.

Koblizek T I, Weiss C, G D Yancopoulos C, Deutsch U, and Risau W. Angiopoietin-1 induces sprouting angiogenesis in vitro. Curr Biol 1998;8:529-532.

Kocher A A, Schuster M D, Szabolcs M J, Takuma S, Burkhoff D, Wang J, Homma S, Edwards N M, Itescu S. Neovascularization of ischemic myocardium by human bone-marrow-derived angioblasts prevents cardiomyocytes apoptosis, reduces remodeling and improves cardiac function. Nat Med 2001;7(4):430-6.

Korff T and Augustin H G. Tensional forces in fibrillar extracellular matrices control directional capillary sprouting. J Cell Sci 1999;112:3249-3259.

Kubota Y, Kleinman H K, Martin G R, and Lawley T J. Role of laminin and basement membrane in the morphological differentiation of human endothelial cells into capillary-like structures. J Cell Biol 1988;107:1589-1598.

Kuzuya M, Satake S, Ramos M A, Kanda S, Koike T, Yoshino K, Ikeda S, and Iguchi A. Induction fo apoptotic cell death in vascular endothelial cells cultured in three-dimensional collagen lattice. Exp Cell Res 1999;248:498-508.

León, E J, Verma, N., Zhang, S., Lauffenburger, D. & Kamm, R. Mechanical properties of a self-assembling oligopeptide matrix. J. Biomaterials Science: Polymer Edl998;9, 297-312.

Leor J, Aboulafia-Etzion S, Dar A, Shapiro L, Barbash I M, Battler A, Granot Y, Cohen S. Bioengineered cardiac grafts: A new approach to repair the infarcted myocardium? Circulation 2000;102(19 Suppl 3):11156-61.

Li R K, Yau T M, Weisel R D, Mickle D A, Sakai T, Choi A, Jia Z Q. Construction of a bioengineered cardiac graft. J Thorac Cardiovasc Surg. 2000;119(2):368-75.

Li R K, Jia Z Q, Weisel R D, Mickle D A, Choi A, Yau T M. Survival and function of bioengineered cardiac grafts. Circulation 1999;100(19 Suppl):1163-9.

Li R K, Jia Z Q, Weisel R D, Mickle D A, Zhang J, Mohabeer M K, Rao V, Ivanov J. Cardiomyocyte transplantation improves heart function. Ann Thorac Surg 1996;62(3):654-60.

Li Z, Bing O H, Long X, Robinson K G, Lakatta E G. Increased cardiomyocyte apoptosis during the transition to heart failure in the spontaneously hypertensive rat. Am J Physiol 1997;272(5 Pt 2):H2313-9.

Maisonpierre P C, Suri C, Jones P F, Bartunkova S, Wiegand S J, Radziejewski C, Compton D, McClain T H, Aldrich T H, Papadopoulos N, Daly T J, Davis S, Sato T N, Yancopoulos G D. Angiopoietin-2, a natural antagonist for Tie2mthat disrupts in vivo angiogenesis. Science 1997;277:55-60.

Mann B K, West J L. Tissue engineering in the cardiovascular system: progress toward a tissue engineered heart. Anat Rec 2001;263(4):367-71.

Montesano R, Orci L, and Vassalli P. In vitro rapid organization of endothelial cells into capillary-like networks is promoted by collagen matrices. J Cell Biol 1983;97: 1648-1652.

Montesano R, Pepper MS, Vassali J D, and Orci L. Phorbol ester induces cultured endothelial cells to invade a fibrin matrix in the presence of fibrinolytic inhibitors. J Cell Physiol 1987;132: 509-516.

Nehls V and Drenckhahn. A novel, microcarrier-based in vitro assay for rapid and reliable quantification of three-dimensional cell migration and angiogenesis. Microvasc Res 1995;50:311-322.

Nehls V and Herrmann R. The configuration of fibrin clots determines capillary morphogenesis and endothelial cell migration. Microvasc Res 1996:51:347-364.

Papadaki M, Bursac N, Langer R, Merok J, Vunjak-Novakovic G, Freed L E. Tissue engineering of functional cardiac muscle: molecular, structural, and electrophysiological studies. Am J Physiol 2001;280(46):H168-178.

Papapetropoulos A, Garcia-Cardena G, Dengler T J, Maisonpierre P C, Yancopoulos G D, Sessa W C. Direct actions of angiopoietin-1 on human endothelium: evidence for network stabilization, cell survival, and interaction with other angiogenic growth factors. Lab Invest 1999;79(2):213-23.

Pecher P, Schumacher B A. Angiogenesis in ischemic human myocardium: clinical results after 3 years. Ann Thorac Surg 2000;69(5):1414-1419.

Pollman M J, Naumovski L, Gibbons G H. Endothelial cell apoptosis in capillary network remodeling. J Cell Physiol 1999;178-359-370.

Quani F, Urbanek K, Beltrami A P, Finato N, Beltrami CA, Nadal-Ginard B, Kajstura J, Leri A, Anversa P. Chimerism of the transplanted heart. N Engl J Med 2002;346:5-15.

Sabbah H N. Apoptotic cell death in heart failure. Cardiovasc Res 2000;45:704-712.

Sakai T, Li R K, Weisel R D, Mickle D A, Kim E J, Tomita S, Jia Z Q, Yau T M. J. Autologous heart cell transplantation improves cardiac function after myocardial injury. Ann Thorac Surg 1999;68(6):2074-80.

Sankar S, Mahooti-Brooks N, Bensen L, McCarthy T L, Centrella M, Madri J A. Modulation of transforming growth factor beta receptor levels on microvascular endothelial cells during in vitro angiogenesis. J Clin Invest 1996;97(6):1436-46.

Satake S, Kuzuya M, Ramos M A, Kanda S, and Iguchi A. Angiogenic stimuli are essential for survival of vascular endothelial cells in three-dimensional collagen lattice. Biochemical and Biophys Res Comm 1998;244:642-646.

Scorsin M, Hagege A A, Marotte F, Mirochnik N, Copin H, Bamoux M, Sabri A, Samuel J L, Rappaport L, Menasche P. Does transplantation of cardiomyocytes improve function of infarcted myocardium? Circulation 1997;96(9 Suppl):II-188-93.

Suzuki K, Murtuza B, Smolenski R T, Sammut I A, Suzuki N, Kaneda Y, Yacoub M H. Cell transplantation for the treatment of acute myocardial infarction using vascular endothelial growth factor-expressing skeletal myoblasts. Circulation 2001;104(12 Suppl 1):1207-12.

Taylor D A, Atkins B Z, Hungspreugs P, Jones T R, Reedy M C, Hutcheson K A, Glower D D, Kraus W E. Regenerating functional myocardium: improved performance after skeletal myoblast transplantation. Nat Med 1998;4(8):929-33.

Toma C, Pittenger M F, Kevin S C, Byrne B J, Kessler P D. Human mesenchymal stem cells differentiate to a cardiomyocyte phenotype in the adult murine heart. Circulation 2002;105:93-98.

Tomita S, Li R K, Weisel R D, Mickle D A G, Kim E J, Sakai T, Jia Z G. Autologous transplantation of bone marrow cells improves damaged heart function. Circulation 1999;100[suppl II]:II-247-II-256.

Vacanti J P & Langer R. Tissue engineering: the design and fabrication of living replacement devices for surgical reconstruction and transplantation. Lancet 1999;354(suppl):S134.

Vailhe B, Ronot X, Tranqui P, Usson Y, and Tranqui L. In vitro angiogenesis is modulated by the mechanical properties of fibrin and is related to αᵥβ₃ integrin localization. In Vitro Cell Dev Biol 1997;33: 763-773.

Vailhe B, Vittet D, Feige J-J. In vitro models of vasculogenesis and angiogenesis. Lab Invest 2001;81:439-452.

van Hinsbergh V W, Collen A, Koolwijk P. Role of fibrin matrix in angiogenesis. Ann N Y Acad Sci 2001;936:426-437.

Van Veen T A B, van Rijen H V M, Opthof T. Cardiac gap junction channels: modulation of expression and channel properties. Cardiovasc Res 2001;51:217-299.

Zhang S, Altman M. Peptide self-assembly in functional polymer science and engineering. Reactive and Functional Polymers 1999:41:91-102.

Zhang S, Holmes T, Lockshin C, and Rich A. Spontaneous assembly of a self-complementary oligopeptide to form a stable macroscopic membrane. PNAS 1993;90:3334-3338.

Zhang S, Holmes T C, DiPersio C M, Hynes R O, Su X, and Rich A. Self-complimentary oligopeptide matrices support mammalian cell attachment. Biomaterials 1995;16:1385-1393.

Zhang S, Yan L, Altman M, Lassie M, Nugent H, Frankel F, Lauffenburger DA, Whitesides G M, and Rich A. Biological surface engineering: a simple system for cell pattern formation. Biomaterials 1999;20:1213-1220.

Zhang, S. (2002) Emerging biological materials through molecular self-assembly. Applied Biochemistry and Biotechnology (in press).

Zimmermann W H, Schneiderbanger K S, Schubert P, Didie M, Munzel F, Heubach J F, Kostin S, Neuhuber W L, Eschenhagen T. Tissue engineering of a differentiated cardiac muscle construct. Circ Res 2002;90:1-9.

1. A composition comprising:
a macroscopic structure comprising amphiphilic peptides, wherein the peptides comprise substantially equal proportions of hydrophobic and hydrophilic amino acids, are complementary and structurally compatible, and are capable of self-assembling into abeta-sheet macroscopic structure; and
cardiovascular system cells.

2. The composition of claim 1, wherein the peptides are capable of self-assembling into beta-sheet filaments that further assemble to form a macroscopic structure or network.

3. The composition of claim 1, wherein the structure has an elastic modulus of between approximately 1 and 10 kilopascals.

4. The composition of claim 1, wherein the cardiovascular system cells comprise vascular endothelial cells.

5. The composition of claim 4, wherein the vascular endothelial cells undergo an angiogenic response on or within the structure.

6. The composition of claim 5, wherein the angiogenic response includes formation of capillary-like structures.

7. The composition of claim 4, wherein the angiogenic response occurs in the absence of externally applied angiogenic factors.

8. The composition of claim 4, wherein the vascular endothelial cells do not undergo significant apoptosis while on or within the structure.

9. The composition of claim 4, wherein the vascular endothelial cells are initially placed on the surface of the structure and subsequently migrate into the structure.

10. The composition of claim 4, wherein the vascular endothelial cells are placed on the surface of the structure at a density of approximately 2×10⁴ cells/cm² and 8×10⁴ cells/cm².

11. The composition of claim 4, further comprising one or more additional cell types.

12. The composition of claim 11, wherein the one or more additional cell types comprises cardiac myocytes.

13. The composition of claim 11, wherein the one or more additional cell types comprises cardiac myoblasts.

14. The composition of claim 11, wherein the one or more additional cell types comprises one or more cell types selected from the group consisting of bone marrow cells, periosteal cells, perichondrial cells, fibroblasts, skeletal myoblasts or myocytes, neuronal cells, hippocampal cells, epidermal cells, non-vascular endothelial cells, smooth muscle cells, keratinocytes, basal cells, spinous cells, granular cells, embryonic stem cells, lung cells, immune system cells, ovarian cells, pancreatic cells, cervical cells, liver cells, or foreskin cells.

15. The composition of claim 1, wherein the cells comprise fetal or neonatal cells.

16. The composition of claim 1, wherein the cells comprise adult cells.

17. The composition of claim 1, wherein the cells are human cells.

18. The composition of claim 1, wherein the peptide is selected from the group consisting of RAD16-11, KFE12, and KLD12.

19. The composition of claim 1, wherein the peptides are dissolved in a solution substantially free of electrolytes at a concentration of between 0.5% and 2% inclusive, weight/volume, prior to self-assembly, or wherein the final concentration of the peptides following self-assembly is between 1 and 10 mg/ml inclusive.

20. The composition of claim 1, further comprising a growth or differentiation factor.

21. The composition of claim 1, wherein the macroscopic structure is a nanoscale environment structure.

22. A composition comprising:
a macroscopic nanoscale environment structure; and
cardivascular system cells.

23. The composition of claim 22, wherein the cardiovascular system cells comprise vascular endothelial cells.

24. The composition of claim 22, wherein the cardiovascular system cells comprise cardiac myocytes.

25. A composition comprising:
a three-dimensional peptide gel matrix with vascular endothelial cell networks, wherein the three-dimensional peptide gel matrix comprises amphiphilic peptides, wherein the peptides comprise substantially equal proportions of hydrophobic and hydrophilic amino acids, are complementary and structurally compatible, and are capable of self-assembling into beta-sheet macroscopic structure.

26. The composition of claim 25, wherein the peptides are capable of self-assembling into beta-sheet filaments that further assemble to form a macroscopic structure or network.

27. The composition of claim 25, further comprising cardiac myocytes.

28. The composition of claim 27, wherein the cardiac myocytes exhibit coordinated cell contractions.

29. The composition of claim 25, further comprising stem cells.

30. The composition of claim 25, wherein the stem cells differentiate into cardiovascular system cells.

31. A composition comprising:
a pre-vascularized scaffold comprising vascular endothelial cells cultured on or in a self-assembling peptide gel structure.

32. A method of culturing cells other than vascular endothelial cells comprising steps of:
contacting the cells with the pre-vascularized scaffold of claim 31.

33. A method of culturing cells comprising:
providing cells including cardiovascular system cells; and
contacting a plurality of the cells with a cell culture material comprising amphiphilic peptides, wherein the peptides comprise substantially equal proportions of hydrophilic and hydrophobic amino acids, are complementary and structurally compatible, and are capable of self-assembling into a beta-sheet macroscopic structure.

34. The method of claim 33, wherein the peptides self-assemble into beta-sheet filaments that further assemble to form a macroscopic structure or network.

35. The method of claim 33, wherein the cells comprise cells of one or more types selected from the group consisting of endothelial cells, smooth muscle cells and fibroblasts, where the cells need not necessarily be of cardiovascular system origin.

36. The method of claim 33 wherein the contacting comprises placing the cardiovascular system cells on the surface of the material.

37. The method of claim 33 wherein the contacting comprises encapsulating the cardiovascular system cells in the material.

38. The method of claim 33 wherein the cardiovascular system cells comprise vascular endothelial cells.

39. The method of claim 33 wherein the cardiovascular system cells are derived from a subject.

40. The method of claim 33 further comprising culturing the cardiovascular system cells in vitro prior to contacting them with the cell culture material.

41. A method of culturing cells other than vascular endothelial cells comprising steps of:
contacting the cells with a three-dimensional peptide gel matrix with vascular endothelial cell networks, wherein the three-dimensional peptide gel matrix comprises amphiphilic peptides, wherein the peptides comprise substantially equal proportions of hydrophobic and hydrophilic amino acids, are complementary and structurally compatible, and are capable of self-assembling into a beta-sheet macroscopic structure.

42. The method of claim 41, wherein the cells comprise cardiac myocytes.

43. The method of claim 41, wherein the cells comprise bone marrow cells, periosteal cells, perichondrial cells, fibroblasts, skeletal myoblasts or myocytes, cardiac myoblasts, neuronal cells, hippocampal cells, epidermal cells, non-vascular endothelial cells, smooth muscle cells, keratinocytes, basal cells, spinous cells, granular cells, embryonic stem cells, lung cells, immune system cells, ovarian cells, pancreatic cells, cervical cells, liver cells, or foreskin cells.

44. A method of treating a subject for a condition affecting the cardiovascular system comprising steps of:
identifying a subject in need of treatment; and
administering a composition comprising cardiovascular system cells on or in a macroscopic structure comprising amphiphilic peptides, wherein the peptides comprise substantially equal proportions of hydrophilic and hydrophobic amino acids, are complementary and structurally compatible, and are capable of self-assembling into a beta-sheet macroscopic structure.

45. The method of claim 44, wherein the peptides self-assemble to form a gel prior to administration to the subject.

46. The method of claim 44, wherein the peptides self-assemble to form a gel in vivo after administration to the subject.

47. The method of claim 44, wherein the cardiovascular system cells comprise vascular endothelial cells.

48. The method of claim 44, wherein the cardiovascular system cells are derived from the subject.

49. A method of treating a subject for a condition involving reduced functional or structural activity of an organ or tissue comprising steps of:
identifying a subject in need of treatment for a condition involving reduced functional or structural activity of an organ or tissue; and
administering, to the subject a composition comprising a three-dimensional peptide gel matrix with vascular endothelial cell networks, wherein the three-dimensional peptide gel matrix comprises amphiphilic peptides, wherein the peptides comprise substantially equal proportions of hydrophobic and hydrophilic amino acids, are complementary and structurally compatible, and are capable of self-assembling into a beta-sheet macroscopic structure.

50. The method of claim 49, wherein the cells forming the vascular endothelial cell networks are derived from the subject.

51. The method of claim 49, wherein the composition further comprises at least one cell type characteristic of the organ or tissue having reduced functional or structural activity.

52. A culture kit comprising:
(a) amphiphilic peptides, wherein the peptides comprise substantially equal proportions of hydrophilic and hydrophobic amino acids, are complementary and structurally compatible, and are capable of self-assembling into a beta-sheet macroscopic structure;
(b) instructions for initiating self-assembly of the peptides into a macroscopic structure; and
(c) at least one element selected from the group consisting of: a population of cardiovascular system cells, cell or tissue culture medium, a predetermined amount of a growth factor, a predetermined amount of an electrolyte, instructions for culturing cells on or within a peptide hydrogel structure and for other uses of the system, a vessel in which cell culture may be performed, a liquid in which the peptide can be dissolved, an electrolyte for initiating peptide self-assembly, and one or more growth or differentiation factors.


SEQUENCE LISTING (30 sequences)

SEQ ID NO: 1 - protein, 16 aa, Artificial Sequence
  Description of Artificial SequenceSequences are truly artificial. They were created by inventors,inspired by partial sequence (amino acids 310-325) of zuotin protein from S. cerevisiae.
RADARADARADARADA

SEQ ID NO: 2 - protein, 16 aa, Artificial Sequence
  Description of Artificial SequenceSequences are truly artificial. They were created by inventors, inspired by partial sequence (amino acids 310-325) of zuotin protein from S. cerevisiae.
RADARGDARADARGDA

SEQ ID NO: 3 - protein, 8 aa, Artificial Sequence
  Description of Artificial SequenceSequences are truly artificial. They were created by inventors, inspired by partial sequence (amino acids 310-325) of zuotin protein from S. cerevisiae.
RADARADA

SEQ ID NO: 4 - protein, 16 aa, Artificial Sequence
  Description of Artificial SequenceSequences are truly artificial. They were created by inventors, inspired by partial sequence (amino acids 310-325) of zuotin protein from S. cerevisiae.
RARADADARARADADA

SEQ ID NO: 5 - protein, 8 aa, Artificial Sequence
  Description of Artificial SequenceSequences are truly artificial. They were created by inventors, inspired by partial sequence (amino acids 310-325) of zuotin protein from S. cerevisiae.
RARADADA

SEQ ID NO: 6 - protein, 16 aa, Artificial Sequence
  Description of Artificial SequenceSequences are truly artificial. They were created by inventors, inspired by partial sequence (amino acids 310-325) of zuotin protein from S. cerevisiae.
AEAKAEAKAEAKAEAK

SEQ ID NO: 7 - protein, 8 aa, Artificial Sequence
  Description of Artificial SequenceSequences are truly artificial. They were created by inventors, inspired by partial sequence (amino acids 310-325) of zuotin protein from S. cerevisiae.
AEAKAEAK

SEQ ID NO: 8 - protein, 16 aa, Artificial Sequence
  Description of Artificial SequenceSequences are truly artificial. They were created by inventors, inspired by partial sequence (amino acids 310-325) of zuotin protein from S. cerevisiae.
RAEARAEARAEARAEA

SEQ ID NO: 9 - protein, 8 aa, Artificial Sequence
  Description of Artificial SequenceSequences are truly artificial. They were created by inventors, inspired by partial sequence (amino acids 310-325) of zuotin protein from S. cerevisiae.
RAEARAEA

SEQ ID NO: 10 - protein, 16 aa, Artificial Sequence
  Description of Artificial SequenceSequences are truly artificial. They were created by inventors, inspired by partial sequence (amino acids 310-325) of zuotin protein from S. cerevisiae.
KADAKADAKADAKADA

SEQ ID NO: 11 - protein, 8 aa, Artificial Sequence
  Description of Artificial SequenceSequences are truly artificial. They were created by inventors, inspired by partial sequence (amino acids 310-325) of zuotin protein from S. cerevisiae.
KADAKADA

SEQ ID NO: 12 - protein, 16 aa, Artificial Sequence
  Description of Artificial SequenceSequences are truly artificial. They were created by inventors, inspired by partial sequence (amino acids 310-325) of zuotin protein from S. cerevisiae.
AEAEAHAHAEAEAHAH

SEQ ID NO: 13 - protein, 8 aa, Artificial Sequence
  Description of Artificial SequenceSequences are truly artificial. They were created by inventors, inspired by partial sequence (amino acids 310-325) of zuotin protein from S. cerevisiae.
AEAEAHAH

SEQ ID NO: 14 - protein, 16 aa, Artificial Sequence
  Description of Artificial SequenceSequences are truly artificial. They were created by inventors, inspired by partial sequence (amino acids 310-325) of zuotin protein from S. cerevisiae.
FEFEFKFKFEFEFKFK

SEQ ID NO: 15 - protein, 8 aa, Artificial Sequence
  Description of Artificial SequenceSequences are truly artificial. They were created by inventors, inspired by partial sequence (amino acids 310-325) of zuotin protein from S. cerevisiae.
FEFKFEFK

SEQ ID NO: 16 - protein, 16 aa, Artificial Sequence
  Description of Artificial SequenceSequences are truly artificial. They were created by inventors, inspired by partial sequence (amino acids 310-325) of zuotin protein from S. cerevisiae.
LELELKLKLELELKLK

SEQ ID NO: 17 - protein, 8 aa, Artificial Sequence
  Description of Artificial SequenceSequences are truly artificial. They were created by inventors, inspired by partial sequence (amino acids 310-325) of zuotin protein from S. cerevisiae.
LELELKLK

SEQ ID NO: 18 - protein, 16 aa, Artificial Sequence
  Description of Artificial SequenceSequences are truly artificial. They were created by inventors, inspired by partial sequence (amino acids 310-325) of zuotin protein from S. cerevisiae.
AEAEAKAKAEAEAKAK

SEQ ID NO: 19 - protein, 8 aa, Artificial Sequence
  Description of Artificial SequenceSequences are truly artificial. They were created by inventors, inspired by partial sequence (amino acids 310-325) of zuotin protein from S. cerevisiae.
AEAEAKAK

SEQ ID NO: 20 - protein, 16 aa, Artificial Sequence
  Description of Artificial SequenceSequences are truly artificial. They were created by inventors, inspired by partial sequence (amino acids 310-325) of zuotin protein from S. cerevisiae.
KAKAKAKAEAEAEAEA

SEQ ID NO: 21 - protein, 16 aa, Artificial Sequence
  Description of Artificial SequenceSequences are truly artificial. They were created by inventors, inspired by partial sequence (amino acids 310-325) of zuotin protein from S. cerevisiae.
AEAEAEAEAKAKAKAK

SEQ ID NO: 22 - protein, 16 aa, Artificial Sequence
  Description of Artificial SequenceSequences are truly artificial. They were created by inventors, inspired by partial sequence (amino acids 310-325) of zuotin protein from S. cerevisiae.
RARARARADADADADA

SEQ ID NO: 23 - protein, 16 aa, Artificial Sequence
  Description of Artificial SequenceSequences are truly artificial. They were created by inventors, inspired by partial sequence (amino acids 310-325) of zuotin protein from S. cerevisiae.
ADADADADARARARAR

SEQ ID NO: 24 - protein, 16 aa, Artificial Sequence
  Description of Artificial SequenceSequences are truly artificial. They were created by inventors, inspired by partial sequence (amino acids 310-325) of zuotin protein from S. cerevisiae.
DADADADARARARARA

SEQ ID NO: 25 - protein, 16 aa, Artificial Sequence
  Description of Artificial SequenceSequences are truly artificial. They were created by inventors, inspired by partial sequence (amino acids 310-325) of zuotin protein from S. cerevisiae.
HEHEHKHKHEHEHKHK

SEQ ID NO: 26 - protein, 16 aa, Artificial Sequence
  Description of Artificial SequenceSequences are truly artificial. They were created by inventors, inspired by partial sequence (amino acids 310-325) of zuotin protein from S. cerevisiae.
ADADADADARARARAR

SEQ ID NO: 27 - protein, 8 aa, Artificial Sequence
  Description of Artificial SequenceSequences are truly artificial. They were created by inventors, inspired by partial sequence (amino acids 310-325) of zuotin protein from S. cerevisiae.
HEHEHKHK

SEQ ID NO: 28 - protein, 20 aa, Artificial Sequence
  Description of Artificial SequenceSequences are truly artificial. They were created by inventors, inspired by partial sequence (amino acids 310-325) of zuotin protein from S. cerevisiae.
VEVEVEVEVEVEVEVEVEVE

SEQ ID NO: 29 - protein, 20 aa, Artificial Sequence
  Description of Artificial SequenceSequences are truly artificial. They were created by inventors, inspired by partial sequence (amino acids 310-325) of zuotin protein from S. cerevisiae.
RFRFRFRFRFRFRFRFRFRF

SEQ ID NO: 30 - protein, 12 aa, Artificial Sequence
  Description of Artificial SequenceSequences are truly artificial. They were created by inventors, inspired by partial sequence (amino acids 310-325) of zuotin protein from S. cerevisiae.
AEAEAEAEAKAK